(19) United States
(12) Patent Application Publication
(10) Pub. No.: US 2024/0175035 A1
(43) Pub. Date: May 30, 2024

(54) COMPOSITIONS AND METHODS FOR TREATING HYPERCHOLESTEROLEMIA

(71) Applicant: EmendoBio Inc., Wilmington, DE (US)

(72) Inventors: Rafi Emmanuel, Ramla (IL); Michal Golan Mashiach, Ness Ziona (IL); Joseph Dicken, Rehovot (IL)

(73) Assignee: EmendoBio Inc., Wilmington, DE (US)

(21) Appl. No.: 18/551,817

(22) PCT Filed: Mar. 22, 2022
(86) PCT No.: PCT/US2022/021263

§ 371 Date: Sep. 21, 2023

Related U.S. Application Data
(60) Provisional application No. 63/304,170, filed on Jan. 28, 2022.
(60) Provisional application No. 63/164,396, filed on Mar. 22, 2021.

(51) Int. Cl. C12N 15/113; A61K 31/7088; A61K 38/46; A61P 3/06; C12N 9/22; C12N 15/11; C12N 15/90

(52) U.S. Cl. CPC C12N 15/1138; A61K 31/7088; A61K 38/465; A61P 3/06; C12N 9/22; C12N 15/11; C12N 15/907; C12N 2310/14; C12N 2310/141; C12N 2310/20; C12N 2310/531; C12N 2800/80

(57) ABSTRACT

RNA molecules comprising a guide sequence portion having 17-50 contiguous nucleotides containing nucleotides in the sequence set forth in any one of SEQ ID NOs: 1-10736 and compositions, methods, and uses thereof.

This application claims the benefit of U.S. Provisional Application Nos. 63/304,170, filed Jan. 28, 2022, and 63/164,396, filed Mar. 22, 2021, the contents of each of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “220321_91709-A-PCT_Sequence_Listing_AWG.txt”, which is 2,288 kilobytes in size, and which was created on Mar. 17, 2022 in the IBM-PC machine format, having an operating system compatibility with MS-Windows, which is contained in the text file filed Mar. 21, 2021 as part of this application.

### BACKGROUND OF INVENTION

Hypercholesterolemia, or high cholesterol, is defined as the presence of high plasma cholesterol levels. Familial hypercholesterolemia (FH) is a hereditary disease primarily due to mutations in the Low-Density Lipoprotein Receptor (LDLR) that lead to elevated cholesterol and premature development of cardiovascular disease.

Low-Density Lipoprotein Receptor (LDLR) binds Low-Density Lipoprotein (LDL), which is the major cholesterol-carrying lipoprotein of plasma, and transports it into cells by endocytosis. In order to be internalized, the receptor-ligand complexes must first cluster into clathrin-coated pits. Elevated levels of LDL cholesterol play a central role in the pathogenesis of coronary heart disease. Defects in the LDL receptor or its regulation result in high levels of LDL and premature cardiovascular disease, as demonstrated by the high impact loss-of function mutations in the LDLR gene that cause familial hypercholesterolemia.

Lifelong lipid-lowering medications such as statins or Ezetimibe are currently available, however, they are often intolerable by some patients and fail to attain desired LDL-C levels. A more recent therapeutic approach is based on subcutaneously injected monoclonal antibodies that transiently inhibit Proprotein Convertase Subtilisin/Kexin type 9 (PCSK9), a protein that promotes LDLR lysosomal degradation. Permanent inhibition of PCSK9 by base editing is currently being developed, but the long-lasting consequences of such irreversible knockdown of a gene that is not even the pathogenic basis of FH remain unknown.

### SUMMARY OF THE INVENTION

Disclosed are approaches for increasing expression of the endogenous LDLR gene or the half-life of its transcript to treat or prevent high cholesterol or hypercholesterolemia (e.g., familial hypercholesterolemia). According to some aspects of the invention, the disclosed approaches are for targeting the 3′ untranslated region (UTR) of the LDLR gene to treat or prevent high cholesterol or hypercholesterolemia (e.g., familial hypercholesterolemia). Without limiting the scope to any mechanism of action, according to some aspects of the invention, the disclosed approaches are for knocking out, removing, truncating, or blocking a region in the 3′ untranslated region (UTR) responsible for restricting LDLR transcription and/or translation in order to enhance its expression and boost low-density lipoprotein cholesterol (LDL-C) uptake. Accordingly, biallelic excision or knockout of regulatory elements binding sites (e.g., miRNA binding sites) in the 3′ UTR of the LDLR gene, preferably in a liver cell such as a hepatocyte, for example, as described herein may be utilized to increase the half-life or expression levels of LDLR molecules, and thereby treat, inhibit, prevent, and/or ameliorate hypercholesterolemia. In some embodiments, at least partial excision of the 3′ UTR of the LDLR gene, as described herein, may be utilized to increase the half-life of the transcript or expression of LDLR molecules, and thereby treat, inhibit, prevent, and/or ameliorate hypercholesterolemia.

In some embodiments, there is provided a method for increasing the expression of an endogenous Low-Density Lipoprotein Receptor (LDLR) gene in a cell, the method comprising modifying the LDLR gene or a transcript encoded by the LDLR gene. In some embodiments, there is provided a method for increasing the expression of an endogenous Low-Density Lipoprotein Receptor (LDLR) gene in a cell, the method comprising modifying, blocking, or removing at least a portion of the 3′ UTR of the LDLR gene or of an LDLR transcript.

In one embodiment, the method comprises
introducing to the cell a composition comprising:
   a CRISPR nuclease or a nucleotide molecule encoding the CRISPR nuclease; and
   an RNA molecule comprising a guide sequence portion having 17-50 nucleotides or
   a nucleotide molecule encoding the same,
wherein a complex of the CRISPR nuclease and the RNA molecule affects a double strand break in an allele of the LDLR gene.

In some embodiments, the LDLR 3′UTR or a portion thereof is excised by the combination of a first double strand break formed by a CRISPR nuclease and a first RNA molecule and a second double strand break formed by a CRISPR nuclease and a second RNA molecule.

As a non-limiting example, a portion of the LDLR 3′UTR may be excised by the combined activity of a first CRISPR complex comprising an OMNI-50 CRISPR nuclease (SEQ ID NO: 10749) and a first RNA molecule comprising a guide sequence portion that comprises SEQ ID NO: 7707 and a second CRISPR complex comprising an OMNI-50 CRISPR nuclease (SEQ ID NO: 10749) and a second RNA molecule comprising a guide sequence portion that comprises SEQ ID NO: 9843.

In another non-limiting example, a portion of the LDLR 3′UTR may be excised by the combined activity of a first CRISPR complex comprising an OMNI-79 CRISPR nuclease (SEQ ID NO: 10744) and a first RNA molecule comprising a guide sequence portion that comprises SEQ ID NO: 7871 and a second CRISPR complex comprising an OMNI-79 CRISPR nuclease (SEQ ID NO: 10744) and a second RNA molecule comprising a guide sequence portion that comprises SEQ ID NO: 9843.

According to embodiments of the present invention, there is provided an RNA molecule comprising a guide sequence portion having 17-50 contiguous nucleotides containing nucleotides in the sequence set forth in any one of SEQ ID Nos: 1-10736.

According to some embodiments of the present invention, there is provided a composition comprising an RNA molecule comprising a guide sequence portion having 17-50 contiguous nucleotides containing nucleotides in the sequence set forth in any one of SEQ ID NOs: 1-10736 and a CRISPR nuclease.

According to some embodiments of the present invention, there is provided a method for increasing the expression of a LDLR gene in a cell, for example, by inactivating a LDLR miRNA binding site in the cell, the method comprising delivering to the cell a composition comprising an RNA molecule comprising a guide sequence portion having 17-50 contiguous nucleotides containing nucleotides in the sequence set forth in any one of SEQ ID NOs: 1-10736 and a CRISPR nuclease. In some embodiments the cell is a liver cell. In some embodiments the cell is a hepatocyte. In some embodiments the cell is a stem cell. In some embodiments, the delivering to the cell is performed in vivo, ex vivo, or in vitro. In some embodiments, the delivery to the cell is performed by in vivo delivery of a lentivirus, adeno-associated virus (AAV) or nanoparticle to the liver. In some embodiments, the method is performed ex vivo and the cell is provided/explanted from an individual patient. In some embodiments, the method further comprises the step of introducing the resulting cell, with at least one modified LDLR allele containing an inactivated miRNA binding site, into the individual patient.

According to some embodiments of the present invention, there is provided a method for treating and/or preventing hypercholesterolemia (e.g., familial hypercholesterolemia), the method comprising delivering to a cell of a subject having or at risk of experiencing hypercholesterolemia a composition comprising an RNA molecule comprising a guide sequence portion having 17-50 contiguous nucleotides containing nucleotides in the sequence set forth in any one of SEQ ID NOS: 1-10736 and a CRISPR nuclease.

According to some embodiments of the present invention, there is provided use of a composition comprising an RNA molecule comprising a guide sequence portion having 17-50 contiguous nucleotides containing nucleotides in the sequence set forth in any one of SEQ ID NOS: 1-10736 and a CRISPR nuclease for inactivating a miRNA binding site of a LDLR allele in a cell, comprising delivering to the cell the composition comprising an RNA molecule comprising a guide sequence portion having 17-50 contiguous nucleotides containing nucleotides in the sequence set forth in any one of SEQ ID NOs: 1-10736 and a CRISPR nuclease.

According to embodiments of the present invention, there is provided a medicament comprising a composition comprising an RNA molecule comprising a guide sequence portion having 17-50 contiguous nucleotides containing nucleotides in the sequence set forth in any one of SEQ ID NOs: 1-10736 and a CRISPR nuclease, wherein the medicament is administered by delivering the composition to the cell.

According to some embodiments of the present invention, there is provided use of a composition comprising an RNA molecule comprising a guide sequence portion having 17-50 contiguous nucleotides containing nucleotides in the sequence set forth in any one of SEQ ID NOS: 1-10736 and a CRISPR nuclease for treating, ameliorating, or preventing hypercholesterolemia, comprising delivering to a cell of a subject having or at risk of experiencing hypercholesterolemia the composition.

According to some embodiments of the present invention, there is provided a medicament comprising a composition comprising an RNA molecule comprising a guide sequence portion having 17-50 contiguous nucleotides containing nucleotides in the sequence set forth in any one of SEQ ID NOs: 1-10736 and a CRISPR nuclease for use in treating, ameliorating, or preventing hypercholesterolemia, wherein the medicament is administered by delivering to a cell of a subject having or at risk of experiencing hypercholesterolemia the composition.

According to some embodiments of the present invention, there is provided a kit for increasing LDLR expression in a cell, comprising an RNA molecule comprising a guide sequence portion having 17-50 contiguous nucleotides containing nucleotides in the sequence set forth in any one of SEQ ID NOs: 1-10736, a CRISPR nuclease, and optionally a tracrRNA molecule; and instructions for delivering the RNA molecule, CRISPR nuclease, and optionally the tracrRNA to the cell.

According to some embodiments of the present invention, there is provided a kit for treating or preventing hypercholesterolemia in a subject, comprising an RNA molecule comprising a guide sequence portion having 17-50 contiguous nucleotides containing nucleotides in the sequence set forth in any one of SEQ ID NOs: 1-10736, a CRISPR nuclease, and/or a tracrRNA molecule; and instructions for delivering the RNA molecule, CRISPR nuclease, and optionally the tracrRNA to a cell of a subject having or at risk of hypercholesterolemia.

According to some embodiments of the present invention, there is provided a cell modified according to any one of the methods described herein or modified using any one of the compositions described herein.

### BRIEF DESCRIPTION OF THE DRAWINGS

FIGS. 1A-1H: Functional proof-of-concept of LDLR 3′UTR excision. The 3′ UTR of the LDLR gene was modified using SpCas9 and two single-guide RNAs (sgRNAs) simultaneously. This targeting was performed in HepG2 cells and either excised nearly the entire 3′ UTR region (g1+g9), the miR85 binding area (g6+g7), or a 2.5 kilobase region deletion which was previously shown to upregulate LDLR expression in FH patients (FIG. 1A). All the excised cells showed upregulation of both mRNA (measured by qRT-PCR “Rq” values. FIG. 1B), total protein abundance (measured by western blot, FIG. 1C), and membrane protein abundance (measured by flow cytometry analysis, FIG. 1D). The effect was especially pronounced in cells that underwent excision of the total 3′ UTR. Fluorescent-LDL cholesterol uptake in cells that underwent excision of 3′ UTR by guides g1+g9 was measured by flow cytometry at the indicated time points. An up to 2-fold upregulation of cholesterol uptake at all the time points was demonstrated (LDL-DyLight™ 488—cholesterol uptake, FIG. 1E). Independent 3′UTR LDLR excision experiments were performed with various pairs of sgRNAs (FIG. 1F and FIG. 1G). A cleavage map of the LDLR 3′UTR excision experiments shown in FIG. 1F and FIG. 1G, including the effect of sgRNA pairings on LDLR expression (Rq values), is shown in FIG. 1H. Note that pairings which do not encompass the ddPCR probe site are not detectable with ddPCR.

FIGS. 2A-2D: Heterozygous LDLR-mutant LCL cells from patients: Reduced levels of surface receptor confirmed in both patients. Flow cytometry analysis of membrane LDLR in mutant-patient LCL cell lines vs. WT cells (FIG. 2A). % Expression was calculated as percentage of geometric mean of each cell line out of the corresponding value for WT LCL cells (FIG. 2B). FH patient LCL cells carrying a GM1448 mutation corresponds to a deletion of Gly197 [ΔG197] (FIG. 2C), and a GM1460 mutation corresponds to substitution of tyrosine for cysteine at codon 646 [C646Y] (FIG. 2D).

FIGS. 3A-3D: Excision of LDLR 3′ UTR in Heterozygous FH patient LCL cells leads to upregulation of surface LDLR and increase in cholesterol uptake. The 3′ UTR of the LDLR gene was excised using specific guides (g1 and g9) and SpCas9 in heterozygous FH patient-derived LCL cell lines. Following excision, the cells were analyzed for LDLR expression levels and LDL-cholesterol uptake. FIG. 3A. Excision percentage of LDLR 3′ UTR as measured by ddPCR. FIG. 3B. mRNA levels of LDLR as measured by qRT-PCR. FIG. 3C. LDLR surface protein expression levels as measured by flow cytometry using a specific antibody. FIG. 3D. Uptake of LDL-Dylight-488-cholesterol measured by flow cytometry following a four (4) hour incubation.

FIGS. 4A-4C: Excision of 3′ UTR of LDLR in mouse liver cell line Hepal-6 shows compatible results to human liver HepG2 cells. 3′ UTR of LDLR gene was excised using specific guides (g1m and g6m) and SpCas9 in mouse hepatoma cell line Hepal-6. Following the excision, the cells were analyzed for LDLR expression levels and LDL-cholesterol uptake. FIG. 4A. mRNA levels of LDLR as measured by qRT-PCR. FIG. 4B. LDLR surface protein expression levels as measured by flow cytometry with a specific antibody. FIG. 4C. Uptake of LDL-Dylight-488-cholesterol measured by flow cytometry following a four (4) hour incubation.

FIGS. 5A-5C: LDLR OMNI-79 CRISPR nuclease based guide screen in the 3′UTR region. FIG. 5A. Transfection-based guide screen in Hela cells of guides targeting indicated regions 3′ UTR of LDLR with either WT OMNI-79 or OMNI-79 V5570. Several guides showed high level of editing and are further evaluated as RNP or RNA compositions. FIG. 5B. Transfection-based guide screen in Hela cells of guides targeting indicated regions 3′ UTR of LDLR with OMNI-103. High activity of multiple guide molecules using OMNI-103 CRISPR nuclease. FIG. 5C. Schematic of guide molecule LDLR target sites.

FIGS. 6A-6E: Knock-out of PCSK9 by CRISPR-based editing and its effect on LDLR expression and LDL-uptake in HepG2 cells. FIG. 6A. PCSK9 Editing—The level of editing in HepG2 cells was measured following modification of PCSK9 by specific guide molecules. FIG. 6B. PCSK9 qRT-PCR—The mRNA levels of PCSK9 in HepG2 cells following editing of PCSK9. FIG. 6C. Secreted PCSK9—The secretion of PCSK9 in PCSK9-edited cells compared to WT HepG2 cells was measured by ELISA. FIG. 6D. LDLR Expression—The membrane expression of LDLR was measured in PCSK9-modified cells compared to wild-type and LDLR 3′UTR excised cells. FIG. 6E. LDL-cholesterol Uptake—488-LDL cholesterol uptake was measured in these cells for four (4) hours with or without addition of lovastatin for 16 hours.

FIGS. 7A-7C: In order to identify regions of the LDLR 3′ UTR essential for upregulation of LDLR expression, portions of the LDLR 3′UTR were excised using several different combinations of specific sgRNAs with SpCas9 in the human hepatic carcinoma cell line HepG2. Following excision, the cells were analyzed for both mRNA and surface protein LDLR expression levels. FIG. 7A. The editing percentage of each individual sgRNA was >90% for every guide tested. FIG. 7B. Excision percentage of LDLR 3′ UTR as measured by ddPCR (in quadruplicate, where applicable), mRNA levels of LDLR as measured by qRT-PCR (presented in duplicates), and LDLR membrane protein expression levels as measured by flow cytometry using a LDLR-specific antibody for each of the indicated LDLR 3′ UTR excisions are shown in triplicate. A cleavage map of the LDLR 3′UTR excision experiments shown in FIG. 7A and FIG. 7B, including the effect of sgRNA pairings on LDLR expression (Rq values), is shown in FIG. 7C.

FIGS. 8A-8D: Excision of the LDLR 3′UTR in HepG2 cells by OMNI-50 ribonucleoprotein (RNP) complexes with guides g46 and g79. Briefly, the 3′UTR of the LDLR gene was excised using specific guides and an OMNI-50 CRISPR nuclease in the human cell line HepG2. Following the excision, the cells were analyzed for LDLR expression levels and LDL-cholesterol uptake. FIG. 8A. % Excision of the LDLR 3′UTR according to ddPCR measurement. FIG. 8B. mRNA levels of LDLR as measured by qRT-PCR. FIG. 8C. LDLR membrane protein expression levels as measured by flow cytometry with a LDLR-specific antibody. FIG. 8D. Uptake of LDL-Dylight-488-cholesterol as measured by flow cytometry following 4-hour incubation with or without lovastatin.

FIGS. 9A-9D: Excision of the LDLR 3′UTR in HepG2 cells by introduction of an OMNI-79 ribonucleoprotein (RNP) complex with guides g38 and g79 or introduction of an RNA composition comprising an OMNI-79 encoding mRNA molecule and guides g38 and g79. Briefly, the 3′UTR of the LDLR gene was excised in HepG2 using specific guides and an OMNI-79 CRISPR nuclease. Following the excision, the cells were analyzed for LDLR expression levels and LDL-cholesterol uptake. FIG. 9A. % Excision of the LDLR 3′UTR according to ddPCR measurement. FIG. 9B. mRNA levels of LDLR as measured by qRT-PCR. FIG. 9C. LDLR membrane protein expression levels as measured by flow cytometry with LDLR-specific antibody. FIG. 9D. Uptake of LDL-Dylight-488-cholesterol as measured by flow cytometry following 4-hour incubation with or without lovastatin.

FIG. 10: Schematic of CRISPR-based LDLR 3′UTR excision strategy according to an embodiment of the invention.

### DETAILED DESCRIPTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

In some embodiments of the present invention, a DNA nuclease is utilized to affect a DNA break at a target site to induce cellular repair mechanisms, for example, but not limited to, non-homologous end-joining (NHEJ). During classical NHEJ, two ends of a double-strand break (DSB) site are ligated together in a fast but also inaccurate manner (i.e. frequently resulting in mutation of the DNA at the cleavage site in the form of small insertion or deletions).

As used herein, the term “modified cells” refers to cells in which a double strand break is affected by a complex of an RNA molecule and the CRISPR nuclease as a result of hybridization with the target sequence, i.e. on-target hybridization.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease, either alone or in combination with other RNA molecules, with the targeting sequence serving as the targeting portion of the CRISPR complex. When the molecule having the targeting sequence is present contemporaneously with the CRISPR molecule, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferentially hybridizing a targeting sequence of a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-30, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. In some embodiments, the guide sequence portion is 17-50 nucleotides. In some embodiments, the guide sequence portion is 17-25 nucleotides. In some embodiments, the guide sequence portion is 17-22 nucleotides. In some embodiments, the guide sequence portion is 17-20 nucleotides. In some embodiments, the entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the DNA molecule having the guide sequence portion is present contemporaneously with the CRISPR molecule the RNA molecule is capable of targeting the CRISPR nuclease to the specific target DNA sequence. Each possibility represents a separate embodiment. An RNA molecule can be custom designed to target any desired sequence. Accordingly, a molecule comprising a “guide sequence portion” is a type of targeting molecule. In some embodiments, the guide sequence portion comprises a sequence that is the same as, or differs by no more than 1, 2, 3, 4, or 5 nucleotides from, a guide sequence portion described herein, e.g., a guide sequence set forth in any of SEQ ID NOs: 1-20246. Each possibility represents a separate embodiment. In some of these embodiments, the guide sequence portion is fully complementary to the target sequence, and comprises a sequence that is the same as a sequence set forth in any of SEQ ID NOs:1-20246. Throughout this application, the terms “guide molecule,” “RNA guide molecule,” “guide RNA molecule,” and “gRNA molecule” are synonymous with a molecule comprising a guide sequence portion.

The term “non-discriminatory” as used herein refers to a guide sequence portion of an RNA molecule that targets a specific DNA sequence that is common to all alleles of a gene.

In embodiments of the present invention, an RNA molecule comprises a guide sequence portion having 17-50 contiguous nucleotides containing nucleotides in the sequence set forth in any one of SEQ ID NOs: 1-10736.

The RNA molecule and or the guide sequence portion of the RNA molecule may contain modified nucleotides. Exemplary modifications to nucleotides/polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. An example of a modified polynucleotide is an mRNA containing 1-methyl pseudo-uridine. For examples of modified polynucleotides and their uses, see U.S. Pat. No. 8,278,036, PCT International Publication No. WO/2015/006747, and Weissman and Kariko (2015), hereby incorporated by reference.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 17-50 nucleotides in length and contain 20-22 contiguous nucleotides in the sequence set forth in any one of SEQ ID NOs: 1-10736. In embodiments of the present invention, the guide sequence portion may be less than 22 nucleotides in length. For example, in embodiments of the present invention the guide sequence portion may be 17, 18, 19, 20, or 21 nucleotides in length. In such embodiments the guide sequence portion may consist of 17, 18, 19, 20, or 21 nucleotides, respectively, in the sequence of 17-22 contiguous nucleotides set forth in any one of SEQ ID NOs: 1-10736. For example, a guide sequence portion having 17 nucleotides in the sequence of 17 contiguous nucleotides set forth in SEQ ID NO: 10739 may consist of any one of the following nucleotide sequences (nucleotides excluded from the contiguous sequence are marked in strike-through):

(SEQ ID NO: 10739)
AAAAAAAUGUACUUGGUUCC
17 nucleotide guide sequence 1:
(SEQ ID NO: 10740) [symbol]
AAAAUGUACUUGGUUCC
17 nucleotide guide sequence 2:
(SEQ ID NO: 10741) [symbol]
AAAAAUGUACUUGGUUC [symbol]
17 nucleotide guide sequence 3:
(SEQ ID NO: 10742) [symbol]
AAAAAAUGUACUUGGUU [symbol]
17 nucleotide guide sequence 4:
(SEQ ID NO: 10743)
AAAAAAAUGUACUUGGU

[symbol]

In embodiments of the present invention, the guide sequence portion may be greater than 20 nucleotides in length. For example, in embodiments of the present invention the guide sequence portion may be 21, 22, 23, 24, 25, 26, 27, 28, 29, or 30 nucleotides in length. In such embodiments the guide sequence portion comprises 17-50 nucleotides containing the sequence of 20, 21 or 22 contiguous nucleotides set forth in any one of SEQ ID NOs: 1-10736 and additional nucleotides fully complimentary to a nucleotide or sequence of nucleotides adjacent to the 3′ end of the target sequence, 5′ end of the target sequence, or both.

In embodiments of the present invention a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule. A guide sequence portion, which comprises a nucleotide sequence that is capable of hybridizing to a specific target DNA sequence, and a sequence portion that participates in CRIPSR nuclease binding, e.g. a tracrRNA sequence portion, can be located on the same RNA molecule. Alternatively, a guide sequence portion may be located on one RNA molecule and a sequence portion that participates in CRIPSR nuclease binding, e.g. a tracrRNA portion, may located on a separate RNA molecule. A single RNA molecule comprising a guide sequence portion (e.g. a DNA-targeting RNA sequence) and at least one CRISPR protein-binding RNA sequence portion (e.g. a tracrRNA sequence portion), can form a complex with a CRISPR nuclease and serve as the DNA-targeting molecule. In some embodiments, a first RNA molecule comprising a DNA-targeting RNA portion, which includes a guide sequence portion, and a second RNA molecule comprising a CRISPR protein-binding RNA sequence interact by base pairing to form an RNA complex that targets the CRISPR nuclease to a DNA target site or, alternatively, are fused together to form an RNA molecule that complexes with the CRISPR nuclease and targets the CRISPR nuclease to a DNA target site.

In embodiments of the present invention, a RNA molecule comprising a guide sequence portion may further comprise the sequence of a tracrRNA molecule. Such embodiments may be designed as a synthetic fusion of the guide portion of the RNA molecule and the trans-activating crRNA (tracrRNA). (See Jinek et al., 2012). In such an embodiment, the RNA molecule is a single guide RNA (sgRNA) molecule. Embodiments of the present invention may also form CRISPR complexes utilizing a separate tracrRNA molecule and a separate RNA molecule comprising a guide sequence portion. In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

According to embodiments of the present invention, there is provided a method for increasing the expression of an endogenous Low-Density Lipoprotein Receptor (LDLR) gene in a cell, the method comprising modifying the LDLR gene or a transcript encoded by the LDLR gene.

In some embodiments, the increase of the expression of an endogenous LDLR gene in a cell is measured by an at least 10%, at least 25%, at least 50%, at least 75%, at least 100%, at least 150%, at least 200%, at least 250%, at least 300%, at least 350%, at least 400%, at least 450%, at least 500%, at least 550%, or at least 600% increase in LDLR transcript levels or LDLR protein levels relative to LDLR transcript levels or LDLR protein levels in the cell prior to modification of the LDLR gene. Methods of measuring transcript levels (e.g. qRT-PCR) and protein levels (e.g. antibody staining, flow cytometry) are known in the art.

In some embodiments, the increase of the expression of an endogenous LDLR gene results in an at least 10%, at least 25%, at least 50%, at least 75%, at least 100%, at least 150%, at least 200%, at least 250%, at least 300%, at least 350%, at least 400%, at least 450%, or at least 500% increase in LDL-C uptake by the cell relative to the LDL-C uptake by the cell prior to modification of the LDLR gene.

In some embodiments, the LDLR gene is modified by a CRISPR nuclease, a meganuclease, a transcription activator-like effector nucleases (TALEN), or a zinc finger nuclease (ZFN), or wherein a transcript encoded by the LDLR gene is modified by a small interfering RNA (siRNA), short hairpin RNA (shRNA), or microRNA (miRNA) molecule.

In some embodiments, the 3′ untranslated region (UTR) of the LDLR gene or transcript encoded by the LDLR gene is modified. For example, a transcript of the LDLR gene may be modified by binding of an siRNA molecule to the transcript. Such a modification of the transcript may be performed in order to block accessibility to the 3′ UTR of the transcript.

In some embodiments, the 3′UTR of the LDLR gene is modified by excision of the 3′ UTR or a portion thereof. In some embodiments, a region of the 3′UTR of the LDLR gene is excised. In some embodiments, a 5′ end of the excised region of the 3′ UTR is located at least 1, 2, 5, 10, 15, 20, 30, 40, 50, 60 nucleotides downstream to the end of the LDLR stop codon and a 3′ end of the excised region of the 3′ UTR is located at least 1, 2, 5, 10, 15 20, 25, 30, 40, 50, 60 nucleotides upstream to the endogenous LDLR polyadenylation signal. Each possibility represents a separate embodiment. In some embodiments, the excised region of the 3′ UTR is at least 30 nucleotides downstream to the stop codon of the LDLR gene and at least 30 nucleotides upstream to the polyadenylation signal. In some embodiments, the excised region of the 3′UTR of the LDLR gene comprises an excision of at least a region spanning from 500, 450, 400, 300, 200 nucleotides (also referred to as “base pairs (bp)”) upstream of the polyadenylation signal to 100, 90, 70, 80, 70, 60 nucleotides upstream of the polyadenylation signal. Each possibility represents a separate embodiment. In some embodiments, the excised region of the LDLR 3′UTR comprises an excision of at least a region spanning from 500 nucleotides upstream of the polyadenylation signal to 60 nucleotides upstream of the polyadenylation signal. In some embodiments, the excised region of the 3′UTR of the LDLR gene comprises an excision of at least a region spanning from 800, 900, 1000, 1100, 1200, 1300, 1400, 1500 nucleotides downstream of the stop codon to 200, 100, 90, 70, 60, 50 nucleotides upstream of the polyadenylation signal. Each possibility represents a separate embodiment. In some embodiments, the excised region of the 3′UTR of the LDLR gene comprises an excision of at least a region spanning from 800 nucleotides downstream of the stop codon to 60 nucleotides upstream of the polyadenylation signal. In some embodiments, the excised region of the 3′UTR of the LDLR gene comprises an excision of at least a region spanning from between 100-300 nucleotides downstream of the stop codon to between 170-30 nucleotides upstream of the polyadenylation signal. In some embodiments, the excised region of the 3′UTR of the LDLR gene comprises an excision of at least a region spanning from 175 nucleotides downstream of the stop codon to 70 nucleotides upstream of the polyadenylation signal. In some embodiments, the excised region of the 3′UTR of the LDLR gene comprises an excision of at least a region spanning from 230 nucleotides downstream of the stop codon to 70 nucleotides upstream of the polyadenylation signal.

In some embodiments, the method comprises
introducing to the cell a composition comprising:
   at least one CRISPR nuclease, or a nucleotide molecule encoding a CRISPR nuclease; and
   an RNA molecule, or a DNA molecule encoding the RNA molecule, comprising a guide sequence,
   wherein a complex of the CRISPR nuclease and the RNA molecule affects a double strand break in an allele of the LDLR gene, thereby increasing the expression of the LDLR gene.

In some embodiments, one or more sites involved in repressing LDLR expression, such as a miRNA binding site or an AU Rich region (AUR), located in the 3′ UTR of LDLR are inactivated, which results in increased expression of LDLR.

In some embodiments, the composition is introduced to a cell in a subject or to a cell in culture.

In some embodiments the cell is liver cell. In some embodiments the cell is a hepatocyte. In some embodiments the cell is a stem cell.

In some embodiments, the CRISPR nuclease and the RNA molecule, or a nucleotide molecule (e.g. a DNA molecule) encoding the RNA molecule, are introduced to the cell at substantially the same time or at different times.

In some embodiments, a miRNA binding site or AU Rich region (AUR) of the LDLR gene in the cell is subjected to an insertion or deletion mutation. In some embodiments at least one miRNA binding site or AU Rich region (AUR) is excised entirely from one or both LDLR alleles in a cell.

In some embodiments, the guide sequence portion of the RNA molecule targets a site in the LDLR 3′UTR located downstream to the stop codon or upstream to the polyadenylation signal. In some embodiments, the guide sequence portion of the RNA molecule targets a site in the LDLR 3′UTR located at least 1, 2, 5, 10, 15, 20, 30, 40, 50 nucleotides downstream to the end of the stop codon or at least 1, 2, 5, 10, 15, 20, 25, 30, 40, 50 nucleotides upstream to the polyadenylation signal. Each possibility represents a separate embodiment. In some embodiments, the guide sequence portion of the RNA molecule targets a site in the LDLR 3′UTR located at least 30 nucleotides downstream to the stop codon or at least 30 nucleotides upstream to the polyadenylation signal.

In some embodiments, the guide sequence portion of the RNA molecule targets a miRNA binding site in a LDLR allele.

In some embodiments, guide sequence portion of the RNA molecule targets a miR-85 or miR-148 binding site.

In some embodiments, a miR seed sequence is inactivated as a result of the double strand break in an allele of the LDLR gene.

In some embodiments, the miR seed sequence is UGGUGCUA or CACUGUG.

In some embodiments, the miR seed sequence is a miR-185 or miR-148 seed sequence.

In some embodiments, the complex of the CRISPR nuclease and the RNA molecule affects a double strand break in both alleles of the LDLR gene.

In some embodiments, the guide sequence portion of the RNA molecule comprises 17-50 contiguous nucleotides containing nucleotides in the sequence set forth in any one of SEQ ID NOs: 1-10736.

In some embodiments, the composition introduced to the cell further comprises a second RNA molecule, or a nucleotide molecule (e.g. a DNA molecule) encoding the second RNA molecule, comprising a guide sequence portion.

In some embodiments, a guide sequence portion of the second RNA targets a site in the LDLR 3′UTR located at least 30 nucleotides downstream to the stop codon or at least 30 nucleotides upstream to the polyadenylation signal.

In some embodiments, the guide sequence portion of the second RNA molecule comprises 17-50 contiguous nucleotides containing nucleotides in the sequence set forth in any one of SEQ ID NOs: 1-10736 other than the sequence of the first RNA molecule.

In some embodiments, the LDLR 3′UTR or a portion thereof is excised by a double strand break formed by a CRISPR nuclease and the first RNA molecule and a double strand break formed by a CRISPR nuclease and the second RNA molecule. In some embodiments, a sequence comprising at least a portion of the LDLR 3′UTR sequence is excised. In some embodiments, a sequence comprising the LDLR 3′UTR sequence is excised.

As a non-limiting example, a portion of the LDLR 3′UTR may be excised by the combined activity of a first CRISPR complex comprising an OMNI-50 CRISPR nuclease (SEQ ID NO: 10749) and a first RNA molecule comprising a guide sequence portion that comprises SEQ ID NO: 7707 and a second CRISPR complex comprising an OMNI-50 CRISPR nuclease (SEQ ID NO: 10749) and a second RNA molecule comprising a guide sequence portion that comprises SEQ ID NO: 9843.

In another non-limiting example, a portion of the LDLR 3′UTR may be excised by the combined activity of a first CRISPR complex comprising an OMNI-79 CRISPR nuclease (SEQ ID NO: 10744) and a first RNA molecule comprising a guide sequence portion that comprises SEQ ID NO: 7871 and a second CRISPR complex comprising an OMNI-79 CRISPR nuclease (SEQ ID NO: 10744) and a second RNA molecule comprising a guide sequence portion that comprises SEQ ID NO: 9843.

Accordingly, specific guide sequence portions may be used in combination to excise a portion of the LDLR 3′UTR. The following embodiments provide several non-limiting examples of portions of the LDLR 3′ UTR which may be excised in order to increase the expression of the LDLR gene. The indicated genomic locations listed are based on gnomAD v3 database and UCSC Genome Browser assembly ID: hg38, Sequencing/Assembly provider ID: Genome Reference Consortium Human GRCh38.p12 (GCA_000001405.27). Assembly date: December 2013 initial release; December 2017 patch release 12.

In some embodiments, the excised portion of the LDLR 3′UTR spans from approximately 19:11131463 to 19:11133279. For example, the guide sequence portion of the first RNA molecule may comprise SEQ ID NO: 1120 and the guide sequence portion of the second RNA molecule may comprise SEQ ID NO: 2656 in order to excise such a portion from the LDLR 3′UTR.

In some embodiments, the excised portion of the LDLR 3′UTR spans from approximately 19:11131463 to 19:11133713. For example, the guide sequence portion of the first RNA molecule may comprise SEQ ID NO: 1120 and the guide sequence portion of the second RNA molecule may comprise SEQ ID NO: 796 in order to excise such a portion from the LDLR 3′UTR.

In some embodiments, the excised portion of the LDLR 3′UTR spans from approximately 19:11131463 to 19:11132114. For example, the guide sequence portion of the first RNA molecule may comprise SEQ ID NO: 1120 and the guide sequence portion of the second RNA molecule may comprise SEQ ID NO: 477 in order to excise such a portion from the LDLR 3′UTR.

In some embodiments, the excised portion of the LDLR 3′UTR spans from approximately 19:11131463 to 19:11132115. For example, the guide sequence portion of the first RNA molecule may comprise SEQ ID NO: 1120 and the guide sequence portion of the second RNA molecule may comprise SEQ ID NO: 1913 in order to excise such a portion from the LDLR 3′UTR.

In some embodiments, the excised portion of the LDLR 3′UTR spans from approximately 19:11131516 to 19:11133713. For example, the guide sequence portion of the first RNA molecule may comprise SEQ ID NO: 181 and the guide sequence portion of the second RNA molecule may comprise SEQ ID NO: 796 in order to excise such a portion from the LDLR 3′UTR.

In some embodiments, the excised portion of the LDLR 3′UTR spans from approximately 19:11131557 to 19:11131567. For example, the guide sequence portion of the first RNA molecule may comprise SEQ ID NO: 901 and the guide sequence portion of the second RNA molecule may comprise SEQ ID NO: 1927 in order to excise such a portion from the LDLR 3′UTR.

In some embodiments, the excised portion of the LDLR 3′UTR spans from approximately 19:11133279 to 19:11133713. For example, the guide sequence portion of the first RNA molecule may comprise SEQ ID NO: 2656 and the guide sequence portion of the second RNA molecule may comprise SEQ ID NO: 796 in order to excise such a portion from the LDLR 3′UTR.

In some embodiments, the excised portion of the LDLR 3′UTR spans from approximately 19:11133713 to 19:11132114. For example, the guide sequence portion of the first RNA molecule may comprise SEQ ID NO: 796 and the guide sequence portion of the second RNA molecule may comprise SEQ ID NO: 477 in order to excise such a portion from the LDLR 3′UTR.

In some embodiments, the excised portion of the LDLR 3′UTR spans from approximately 19:11133713 to 19:11132115. For example, the guide sequence portion of the first RNA molecule may comprise SEQ ID NO: 796 and the guide sequence portion of the second RNA molecule may comprise SEQ ID NO: 1913 in order to excise such a portion from the LDLR 3′UTR.

In some embodiments, the excised portion of the LDLR 3′UTR spans from approximately 19:11131467 to 19:11133713. For example, the guide sequence portion of the first RNA molecule may comprise SEQ ID NO: 7871 and the guide sequence portion of the second RNA molecule may comprise SEQ ID NO: 9843 in order to excise such a portion from the LDLR 3′UTR.

In some embodiments, the excised portion of the LDLR 3′UTR spans from approximately 19:11131519 to 19:11133713. For example, the guide sequence portion of the first RNA molecule may comprise SEQ ID NO: 7707 and the guide sequence portion of the second RNA molecule may comprise SEQ ID NO: 9843 in order to excise such a portion from the LDLR 3′UTR.

In some embodiments, the endogenous LDLR polyadenylation signal remains intact.

In some embodiments, the guide sequence portion of the first RNA molecule or second RNA molecule comprises 1, 2, 3, 4, or 5 nucleotide mismatches relative to a fully complementary LDLR target sequence.

In some embodiments, the guide sequence portion comprises 17-50 contiguous nucleotides containing nucleotides in the sequence set forth in any one of SEQ ID NOs: 1-10736 modified to contain 1, 2, 3, 4, or 5 nucleotide mismatches relative to a fully complementary LDLR target sequence.

In some embodiments, the guide sequence portion modified to contain 1, 2, 3, 4, or 5 nucleotide mismatches relative to a fully complementary LDLR target sequence provides higher targeting specificity to the complex of the CRISPR nuclease and the first RNA molecule relative to a guide sequence portion that has higher complementarity to the mutant allele of the LDLR gene.

In some embodiments, the increase of the expression of an endogenous LDLR gene in a cell is measured by an at least 10%, at least 25%, at least 50%, at least 75%, at least 100%, at least 150%, at least 200%, at least 250%, at least 300%, at least 350%, at least 400%, at least 450%, at least 500% increase, at least 550% increase, or at least 600% increase in LDLR transcript levels or LDLR protein levels relative to LDLR transcript levels or LDLR protein levels in the cell prior to modification of the LDLR gene.

In some embodiments, the increase of the expression of an endogenous LDLR gene results in an at least 10%, at least 25%, at least 50%, at least 75%, at least 100%, at least 150%, at least 200%, at least 250%, at least 300%, at least 350%, at least 400%, at least 450%, or at least 500% increase in LDL-C uptake by the cell relative to the LDL-C uptake by the cell prior to modification of the LDLR gene.

According to embodiments of the present invention, there is provided a composition comprising an RNA molecule which comprises 17-50 contiguous nucleotides containing nucleotides in the sequence set forth in any one of SEQ ID NOs: 1-10736, or any one of SEQ ID NOs: 1-10736 modified to contain 1, 2, 3, 4, or 5 nucleotide mismatches relative to a fully complementary LDLR target sequence, or a DNA molecule encoding the RNA molecule.

In some embodiments, the composition further comprises a CRISPR nuclease, or a nucleotide molecule encoding a CRISPR nuclease.

In some embodiments, the composition further comprises a tracrRNA molecule.

In some embodiments, the composition further comprises a second RNA molecule, or a DNA molecule encoding the second RNA molecule, comprising a guide sequence portion.

In some embodiments, a guide sequence portion of the second RNA targets a site in the LDLR 3′UTR located at least 30 nucleotides downstream to the stop codon or at least 30 nucleotides upstream to the polyadenylation signal.

In some embodiments, the guide sequence portion of the second RNA molecule comprises 17-50 contiguous nucleotides containing nucleotides in the sequence set forth in any one of SEQ ID NOs: 1-10736 other than the sequence of the first RNA molecule, or any one of SEQ ID NOs: 1-10736 other than the sequence of the first RNA molecule modified to contain 1, 2, 3, 4, or 5 nucleotide mismatches relative to a fully complementary LDLR target sequence.

According to some embodiments of the present invention, there is provided a cell modified according to any one of the methods described herein or modified using any one of the compositions described herein.

According to some embodiments of the present invention, there is provided a medicament comprising the composition of any one of the embodiments described herein for use in inactivating a LDLR allele in a cell, wherein the medicament is administered by delivering to the cell the composition.

According to some embodiments of the present invention, there is provided a use of the composition of any one of the embodiments described herein, or a modified cell described herein, for treating, ameliorating, or preventing hypercholesterolemia, comprising delivering the composition or modified cell to a subject experiencing or at risk of experiencing hypercholesterolemia.

According to some embodiments of the present invention, there is provided a medicament comprising the composition of any one of the embodiments described herein, or a modified cell described herein, for use in treating, ameliorating, or preventing hypercholesterolemia, wherein the medicament is administered by delivering the composition or modified to a subject experiencing or at risk of experiencing hypercholesterolemia.

According to some embodiments of the present invention, there is provided a kit for increasing LDLR expression in a cell, comprising the composition of any one of the embodiments described herein and instructions for delivering the composition to the cell.

According to some embodiments of the present invention, there is provided a kit for treating or preventing hypercholesterolemia in a subject, comprising the composition of any one of the embodiments described herein, or a modified cell described herein, and instructions for delivering the composition or modified cell to a subject experiencing or at risk of experiencing hypercholesterolemia.

According to embodiments of the present invention, there is provided use of any one of the compositions or modified cells described herein for treating, ameliorating, or preventing hypercholesterolemia (e.g., familial hypercholesterolemia), comprising delivering the composition or modified cells to a subject experiencing or at risk of experiencing hypercholesterolemia (e.g., familial hypercholesterolemia).

According to embodiments of the present invention, there is provided a medicament comprising any one of the compositions or modified cells described herein for treating, ameliorating, or preventing hypercholesterolemia (e.g., familial hypercholesterolemia), such that the medicament is administered by delivering the composition or modified cell to a subject experiencing or at risk of experiencing hypercholesterolemia (e.g., familial hypercholesterolemia).

According to embodiments of the present invention, there is provided an RNA molecule for use in modifying a cell (e.g. a liver cell or hepatocyte) to treat, ameliorate, or prevent hypercholesterolemia (e.g., familial hypercholesterolemia) in a subject. The RNA molecule may be delivered to the cell ex vivo, in vitro, or in vivo.

According to embodiments of the present invention, there is provided a kit for inactivating a miRNA binding site and/or AU Rich region (AUR) of a LDLR allele in a cell, comprising any one of the compositions described herein and instructions for delivering the composition to the cell.

According to embodiments of the present invention, there is provided a kit for treating, ameliorating, or preventing hypercholesterolemia (e.g., familial hypercholesterolemia) in a subject, comprising any one of the compositions described herein and instructions for delivering the composition to a subject experiencing or at risk of experiencing hypercholesterolemia (e.g., familial hypercholesterolemia).

According to embodiments of the present invention, there is provided a composition comprising an RNA molecule which comprises 17-50 contiguous nucleotides containing nucleotides in the sequence set forth in any one of SEQ ID NOs: 1-10736, or any one of SEQ ID NOs: 1-10736 modified to contain 1, 2, 3, 4, or 5 nucleotide mismatches relative to a fully complementary LDLR target sequence.

In some embodiments, the composition further comprises a CRISPR nuclease or a functional derivative thereof.

In some embodiments, the composition further comprises a tracrRNA molecule.

According to embodiments of the present invention, there is provided a gene editing composition comprising an RNA molecule comprising a guide sequence portion having 17-50 contiguous nucleotides containing nucleotides in the sequence set forth in any one of SEQ ID NOS: 1-10736, or any one of SEQ ID NOs: 1-10736 modified to contain 1, 2, 3, 4, or 5 nucleotide mismatches relative to a fully complementary LDLR target sequence, or a nucleotide molecule (e.g. a DNA molecule) encoding the same. In some embodiments, the RNA molecule further comprises a portion having a sequence which binds to a CRISPR nuclease. In some embodiments, the sequence which binds to a CRISPR nuclease is a tracrRNA sequence.

In some embodiments, the RNA molecule further comprises a portion having a tracr mate sequence or a molecule encoding the same.

In some embodiments, the RNA molecule may further comprise one or more linker

portions.

According to embodiments of the present invention, an RNA molecule may be up to 1000, 900, 800, 700, 600, 500, 450, 400, 350, 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 100 up to 500 nucleotides in length, 100 up to 400 nucleotides in length, 200 up to 300 nucleotides in length, 100 to 200 nucleotides in length, or 150 up to 250 nucleotides in length. Each possibility represents a separate embodiment.

According to some embodiments of the present invention, the composition further comprises a tracrRNA molecule.

In some embodiments, the composition further comprises a second RNA molecule or a DNA molecule encoding the second RNA molecule.

In some embodiments, a guide sequence portion of the second RNA targets a site in the LDLR 3′UTR located at least 30 nucleotides downstream to the stop codon or at least 30 nucleotides upstream to the polyadenylation signal.

In some embodiments, the guide sequence portion of the second RNA molecule comprises 17-50 contiguous nucleotides containing nucleotides in the sequence set forth in any one of SEQ ID NOs: 1-10736 other than the sequence of the first RNA molecule, or any one of SEQ ID NOs: 1-10736 other than the sequence of the first RNA molecule modified to contain 1, 2, 3, 4, or 5 nucleotide mismatches relative to a fully complementary LDLR target sequence.

The compositions and methods of the present disclosure may be utilized for treating, preventing, ameliorating, or slowing progression of hypercholesterolemia.

Any one of, or combination of, the above-mentioned strategies for increasing LDLR expression or increasing LDLR half-life in a cell may be used in the context of the invention.

In some embodiments, the method is utilized for treating a subject at risk for hypercholesterolemia, which is a disease phenotype that is decreased in severity by increased expression of the LDLR gene. In such embodiments, the method results in improvement, amelioration, or prevention of the disease phenotype.

Embodiments of compositions described herein include at least one CRISPR nuclease, RNA molecule(s), and a tracrRNA molecule, being effective in a subject or cells at the same time. The at least one CRISPR nuclease, RNA molecule(s), and tracrRNA may be delivered substantially at the same time or can be delivered at different times but have effect at the same time. For example, this includes delivering the CRISPR nuclease to the subject or cells before the RNA molecule and/or tracrRNA is substantially extant in the subject or cells.

In some embodiments, a human cell is modified by any one of the methods described herein. In some embodiments, the cell is a liver cell. In some embodiments the cell is a hepatocyte. In some embodiments the cell is a stem cell.

### LDLR Editing Strategies

Specific regions of the human LDLR gene that are able to be modified or removed in order to increase LDLR expression have not yet been determined. Previously. Knouff et al. (2001) showed a modest increase in hepatic LDLR mRNA levels in the liver of mice heterozygotes which were modified to replace a mouse LDLR allele with a truncated human LDLR minigene variant. Additionally. Bjornsson et al. (2021) identified a single family containing several carriers of a 2.5 kb deletion mutation spanning the distal region of the LDLR 3′UTR, including the endogenous polyadenylation signal, and extending well beyond the LDLR gene, which appeared to provide a gain-of-function effect. However, strategies to target and remove specific portions of the human LDLR 3′UTR, for example, by targeting CRISPR nucleases to specific sites, and the effect of such mutations on LDLR expression and cholesterol levels have been absent.

The present invention provides methods to increase expression of an LDLR gene, for example, by removing or inactivating regulatory factor sites or excising a portion of the 3′ UTR of one or more LDLR alleles in cells of a subject, thereby treating or preventing hypercholesterolemia (e.g., familial hypercholesterolemia). Accordingly, the provided methods to knockout or inactivate a regulatory factor site within a LDLR allele in a cell may be used to treat, prevent, or ameliorate hypercholesterolemia (e.g., familial hypercholesterolemia). In some embodiments, the regulatory factor site is a miRNA binding site or AU Rich region (AUR).

LDLR editing strategies include, but are not limited to, utilizing an RNA molecule comprising a guide sequence portion which targets a CRISPR nuclease to a microRNA binding site in the 3′UTR of LDLR. For example, the guide sequence portion may target the miR-185 seed sequence or the miR-148 seed sequence. Editing at such a seed site would eliminate the binding of the miRNAs to the 3′UTR, thus upregulating the LDLR mRNA levels and/or LDLR protein level.

Another strategy includes utilizing two RNA molecules, each comprising a unique guide sequence portion which targets a CRISPR nuclease to a sequence in the LDLR 3′UTR. To achieve biallelic excision of at least a portion of the 3′UTR, a first of two targeted sequences may be located downstream to the LDLR stop codon and the second of the two sequences may be located upstream to the polyadenylation signal. Such sequences may be located at least 30 nucleotides downstream to the stop codon or at least 30 nucleotides upstream to the polyadenylation signal. In this fashion, biallelic excision of most of the 3′UTR is achieved. Accordingly, miRNA binding sites or AU Rich regions (AURs) regulatory sites that negatively regulate the expression of LDLR are disabled or removed. In some embodiments, this is achieved while maintaining a truncated 3′UTR with an intact polyadenylation signal, which is essential for the stability and translation efficiency of the transcript. In other embodiments, the polyadenylation signal is also at least partially removed.

Accordingly, one strategy includes utilizing two guide RNA molecules, each comprising a unique guide sequence portion which targets a sequence in the 3′UTR, and at least one CRISPR nuclease to excise a sequence that includes the 3′ UTR of the LDLR gene or at least a portion of the 3′ UTR of the LDLR gene. Such sequences may be located downstream to the polyadenylation signal or upstream to the stop codon. Non-limiting examples of sequences that can be targeted to achieve biallelic excision comprising at least a portion of the 3′UTR or the entire 3′UTR include: 1) a first sequence located upstream to the stop codon and a second sequence located upstream to the polyadenylation signal: 2) a first sequence located downstream to the stop codon and a second sequence located downstream to the polyadenylation signal: and 3) a first sequence located upstream to the stop codon and a second sequence located downstream to the polyadenylation signal.

Any one of the RNA molecules comprising a guide sequence portion may be a single-guide RNA (sgRNA) molecule or a CRISPR RNA (crRNA) molecule. Typically, a crRNA molecule is utilized with a transactivating crRNA (tracrRNA) molecule to target a CRISPR nuclease, as described in the art. Accordingly, each of the indicated strategies can be performed utilizing one or more CRISPR nucleases with one or more single-guide RNA molecules and/or one or more crRNA and tracrRNA molecules, or any combination thereof. The compositions can be introduced to the target cell as one or more of nucleic acid vectors, DNA molecules, RNA molecules, ribonucleoproteins (RNP), or any combination thereof.

### CRISPR Nucleases and PAM Recognition

In some embodiments, the sequence specific nuclease is selected from CRISPR nucleases, or is a functional variant thereof. In some embodiments, the sequence specific nuclease is an RNA guided DNA nuclease. In such embodiments, the RNA sequence which guides the RNA guided DNA nuclease (e.g., Cpf1) binds to and/or directs the RNA guided DNA nuclease to all LDLR alleles in a cell. In some embodiments, the CRISPR complex does not further comprise a tracrRNA. A skilled artisan will appreciate that RNA molecules can be engineered to bind to a target of choice in a genome by commonly known methods in the art.

The term “PAM” as used herein refers to a nucleotide sequence of a target DNA located in proximity to the targeted DNA sequence and recognized by the CRISPR nuclease complex. The PAM sequence may differ depending on the nuclease identity. In addition, there are CRISPR nucleases that can target almost all PAMs. In some embodiments of the present invention, a CRISPR system utilizes one or more RNA molecules having a guide sequence portion to direct a CRISPR nuclease to a target DNA site via Watson-Crick base-pairing between the guide sequence portion and the protospacer on the target DNA site, which is next to the protospacer adjacent motif (PAM), which is an additional requirement for target recognition. The CRISPR nuclease then mediates cleavage of the target DNA site to create a double-stranded break within the protospacer. In a non-limiting example, a type II CRISPR system utilizes a mature crRNA:tracrRNA complex that directs the CRISPR nuclease. e.g. Cas9 to the target DNA the target DNA via Watson-Crick base-pairing between the guide sequence portion of the crRNA and the protospacer on the target DNA next to the protospacer adjacent motif (PAM). A skilled artisan will appreciate that each of the engineered RNA molecule of the present invention is further designed such as to associate with a target genomic DNA sequence of interest next to a protospacer adjacent motif (PAM). e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NRRH for SpCas9-NRRH variant, wherein N is any nucleobase, R is A or G and H is A, C, or T; NRTH for SpCas9-NRTH variant, wherein N is any nucleobase, R is A or G and H is A, C, or T; NRCH for SpCas9-NRCH variant, wherein N is any nucleobase, R is A or G and H is A, C, or T; NG for SpG variant of SpCas9 wherein N is any nucleobase; NG or NA for SpCas9-NG variant of SpCas9 wherein N is any nucleobase; NR or NRN or NYN for SpRY variant of SpCas9, wherein N is any nucleobase, R is A or G and Y is C or T; NNG for Streptococcus canis Cas9 variant (ScCas9), wherein N is any nucleobase; NNNRRT for SaKKH-Cas9 variant of Staphylococcus aureus (SaCas9), wherein N is any nucleobase, and R is A or G; NNNNGATT for Neisseria meningitidis (NmCas9), wherein N is any nucleobase; TTN for Alicyclobacillus acidiphilus Cas12b (AacCas12b), wherein N is any nucleobase; or TTTV for Cpf1, wherein V is A, C or G. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break, either double or single-stranded in nature, at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Patent Publication No. 2015/0211023, incorporated herein by reference.

CRISPR systems that may be used in the practice of the invention vary greatly. CRISPR systems can be a type I, a type II, or a type III system. Non- limiting examples of suitable CRISPR proteins include Cas3, Cas4, Cas5, Cas5e (or CasD), Cas6, Cas6e, Cas6f, Cas7, Cas8al, Cas8a2, Cas8b, Cas8c, Cas9, Casl0,Casl Od, CasF, CasG, CasH, Csyl , Csy2, Csy3, Csel (or CasA), Cse2 (or CasB), Cse3 (or CasE), Cse4 (or CasC), Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, Csxl0, Csxl6, CsaX, Csx3, Cszl, Csxl5,Csfl, Csf2, Csf3, Csf4, and Cul966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculumthermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., SpCas9 D1135E variant, SpCas9 VQR variant, SpCas9 EQR variant, or SpCas9 VRER variant may also be used in the context of the invention.

Thus, an RNA guided DNA nuclease of a CRISPR system, such as a Cas9 protein or modified Cas9 or homolog or ortholog of Cas9, or other RNA guided DNA nucleases belonging to other types of CRISPR systems, such as Cpf1 and its homologs and orthologs, may be used in the compositions of the present invention. Additional CRISPR nucleases may also be used, for example, the nucleases described in PCT International Application Publication Nos. WO2020/223514 and WO2020/223553, which are hereby incorporated by reference.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Derivatives include, but are not limited to, CRISPR nickases, catalytically inactive or “dead” CRISPR nucleases, and fusion of a CRISPR nuclease or derivative thereof to other enzymes such as base editors or retrotransposons. See for example, Anzalone et al. (2019) and PCT International Application No. PCT/US2020/037560.

Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al., 2015).

Thus, an RNA guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl)uridine, 2′-O-methylcytidine, 5-carboxymethylaminomethyl-2-thiouridine, S-carboxymethylaminomethyluridine, dihydrouridine, 2′-O-methylpseudouridine, “beta, D-galactosylqueuosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylqueuosine”, 5-methoxycarbonylmethyl-2-thiouridine, 5-methoxycarbonyImethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-S-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2″-O-methyluridine, wybutosine, “3-(3-amino-3-carboxy-propyl)uridine, (acp3)u”, 2′-0-methyl (M), 3′-phosphorothioate (MS), 3′-thioPACE (MSP), pseudouridine, or 1-methyl pseudo-uridine. Each possibility represents a separate embodiment of the present invention.

In addition to targeting LDLR alleles by a RNA-guided CRISPR nuclease, other means of inhibiting LDLR expression in a target cell include but are not limited to use of a gapmer, shRNA, siRNA, a customized TALEN, meganuclease, or zinc finger nuclease, a small molecule inhibitor, and any other method known in the art for reducing or eliminating expression of a gene in a target cell. See, for example, U.S. Pat. Nos. 6,506,559; 7,560,438; 8,420,391; 8,552,171; 7,056,704; 7,078,196; 8,362,231; 8,372,968; 9,045,754; and PCT International Publication Nos. WO/2004/067736; WO/2006/097853; WO/2003/087341; WO/2000/0415661; WO/2003/080809; WO/2010/079430; WO/2010/079430; WO/2011/072246; WO/2018/057989; and WO/2017/164230, the entire contents of each of which are incorporated herein by reference.

Advantageously, the guide RNA molecules presented herein provide improved efficiency when complexed with a CRISPR nuclease in a cell relative to other guide RNA molecules. These specifically designed sequences may also be useful for identifying LDLR target sites for other nucleotide targeting-based gene-editing or gene-silencing methods, for example, siRNA, TALENS, meganucleases or zinc-finger nucleases.

### Delivery to Cells

Any one of the compositions described herein may be delivered to a target cell by any suitable means. RNA molecule compositions of the present invention may be targeted to any cell which contains and/or expresses a LDLR allele, such as a mammalian liver cell, hepatocyte, or stem cell. For example, in one embodiment the RNA molecule specifically targets LDLR alleles in a target cell and the target cell is a liver cell, hepatocyte, or stem cell. The delivery to the cell may be performed in vivo, ex vivo, or in vitro. The delivery may be in vivo delivery of a composition packaged in a lentivirus, adeno-associated virus (AAV), or nanoparticle to the liver of a subject, or a liver cell of the subject. The delivery may be ex vivo to a cell of the subject, for example, a liver cell, hepatocyte, or stem cell isolated from the subject. Further, the nucleic acid compositions described herein may be delivered to a cell as one or more of DNA molecules, RNA molecules, ribonucleoproteins (RNP), nucleic acid vectors, or any combination thereof.

In some embodiments, in vivo delivery methods of the compositions described herein include delivery by a lentivirus, adeno-associated virus (AAV) or nanoparticle. In some embodiments, in vivo delivery methods of the compositions described herein include delivery by a lentivirus, adeno-associated virus (AAV), naked RNA, or nanoparticle, such as a lipid nanoparticle. The composition may be in the form of an RNA composition. The composition may be in the form of a ribonucleoprotein (RNP) composition. Accordingly, the delivery can be performed in vivo to liver cells within a subject, for example, a subject suffering from hypercholesterolemia.

In some embodiments, any one of the compositions described herein is delivered to a cell ex-vivo. In some embodiments, the cell is a stem cell. In some embodiments, the cell is a liver cell. In some embodiments, the cell is a hepatocyte. In some embodiments, the cell is an iPS-derived hepatocyte. The composition may be delivered to the cell by any known ex-vivo delivery method, including but not limited to, electroporation, viral transduction, nanoparticle delivery, liposomes, etc. The composition may be in the form of an RNP composition. Additional detailed delivery methods are described throughout this section.

In some embodiments, option a liver lobe or hepatocyte cells are obtained from a subject, for example, by performing a biopsy. The obtained cells may then be genetically modified by ex vivo delivery of a composition described herein. The modified cells may be re-introduced to the subject by performing a lobe transplantation or hepatocyte transplantation.

In some embodiments, the RNA molecule comprises a chemical modification. Non-limiting examples of suitable chemical modifications include 2′-0-methyl (M), 2′-0-methyl, 3′phosphorothioate (MS) or 2′-0-methyl, 3′thioPACE (MSP), pseudouridine, and 1-methyl pseudo-uridine. Each possibility represents a separate embodiment of the present invention.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992); Nabel & Felgner (1993); Mitani & Caskey (1993); Dillon (1993); Miller (1992); Van Brunt (1988); Vigne (1995); Kremer & Perricaudet (1995); Haddada et al. (1995); and Yu et al. (1994).

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus). (See, e.g., Chung et al., 2006). Sonoporation using, e.g., the Sonitron 2000 system (Rich-Mar), can also be used for delivery of nucleic acids. Cationic-lipid mediated delivery of proteins and/or nucleic acids is also contemplated as an in vivo, ex vivo, or in vitro delivery method. (See Zuris et al. (2015); see also Coelho et al. (2013); Judge et al. (2006); and Basha et al. (2011)).

Non-viral vectors, such as transposon-based systems e.g. recombinant Sleeping Beauty transposon systems or recombinant Piggy Bac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa.RTM. Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam.™, Lipofectin.™. and Lipofectamine.™. RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., (1995); Behr et al., (1994); Remy et al. (1994); Gao and Huang (1995); Ahmad and Allen (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al., 2009).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992); Johann et al. (1992); Sommerfelt et al. (1990); Wilson et al. (1989); Miller et al. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (See Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors. (Ellem et al., (1997); Dranoff et al., 1997).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, for example by systemic administration (e.g., intravitreal, intravenous, intraperitoneal, intramuscular, subdermal, or intracranial infusion) or topical application.

Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, optionally after selection for cells which have incorporated the vector. A non-limiting exemplary ex vivo approach may involve removal of tissue (e.g., peripheral blood, bone marrow, and spleen) from a patient for culture, nucleic acid transfer to the cultured cells (e.g., hematopoietic stem cells), followed by grafting the cells to a target tissue (e.g., bone marrow, and spleen) of the patient. In some embodiments, the stem cell or hematopoietic stem cell may be further treated with a viability enhancer.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney, “Culture of Animal Cells, A Manual of Basic Technique and Specialized Applications (6th edition, 2010) and the references cited therein for a discussion of how to isolate and culture cells from patients).

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

### Examples of RNA Guide Sequences Which Specifically Target Alleles of LDLR Gene

Although a large number of guide sequences can be designed to target the LDLR gene, the nucleotide sequences described in Table 1 and are identified by SEQ ID NOs: 1-10736 were specifically selected to effectively implement the methods set forth herein.

Table 1 shows guide sequences designed for use as described in the embodiments above to associate with LDLR alleles. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9. VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), SpRY (PAM SEQ: NRN or NYN), NmCas9WT (PAM SEQ: NNNNGATT), Cpf1 (PAM SEQ: TTTV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

TABLE 1
Guide sequences designed to
associate with specific LDLR gene targets

| | SEQ ID | SEQ ID | SEQ ID |
|---|---|---|---|
| | NOS: | NOs: | NOS: |
| | of 20 base | of 21 base | of 22 base |
| Target | guides | guides | guides |
| 19:11131280-19:11133820 | 1-3564 | 3565-7118 | 7119-10735 |
| LDLR 3'UTR, including | | | |
| 30 nt from the stop | | | |
| codon and 30 nt upstream | | | |
| to the polyadenylation site | | | |

The indicated locations listed in column 1 of the Table 1 are based on gnomAD v3 database and UCSC Genome Browser assembly ID: hg38, Sequencing/Assembly provider ID: Genome Reference Consortium Human GRCh38.p12 (GCA_000001405.27). Assembly date: December 2013 initial release; December 2017 patch release 12.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1: General LDLR Modification Anaylsis

Guide sequences comprising 17-50 contiguous nucleotides containing nucleotides in the sequence set forth in any one of SEQ ID NOs: 1-10736 are screened for high on target activity using SpCas9 in human cells. On target activity is determined by DNA capillary electrophoresis analysis.

### Example 2: LDLR 3′ UTR Modification Proof of Concept

Familial hypercholesterolemia (FH), which is characterized by lifelong elevation of LDL-C, is caused by pathogenic mutations in the LDLR gene in 85-90% of cases (L. Jiang et al, 2015; A. K. Soutar and R. P. Naoumova, 2007).

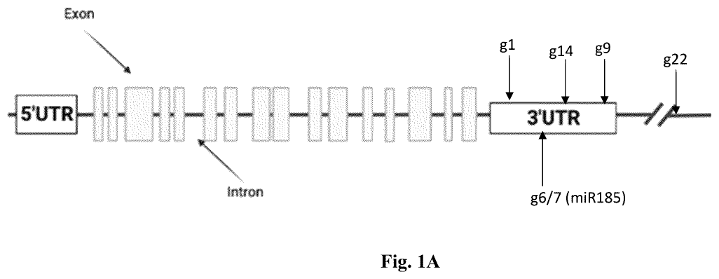

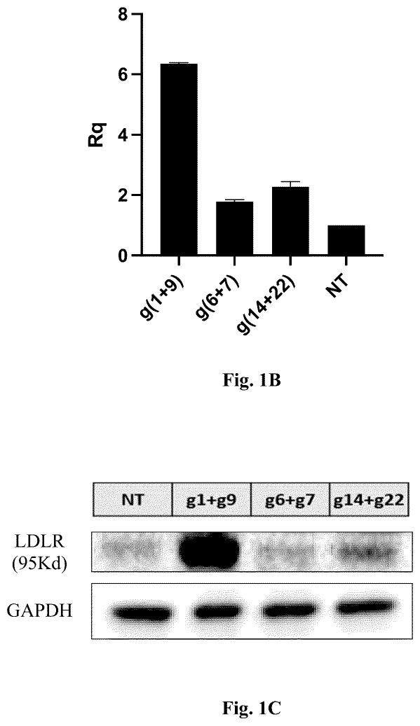

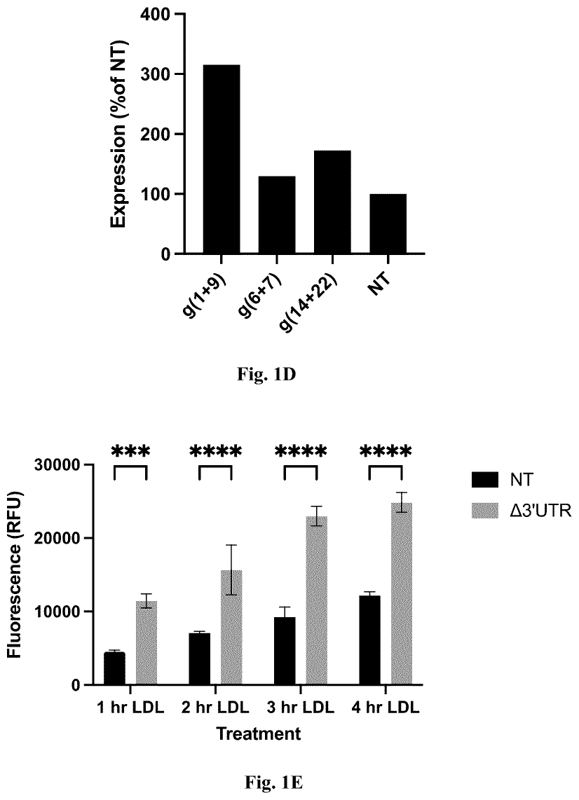

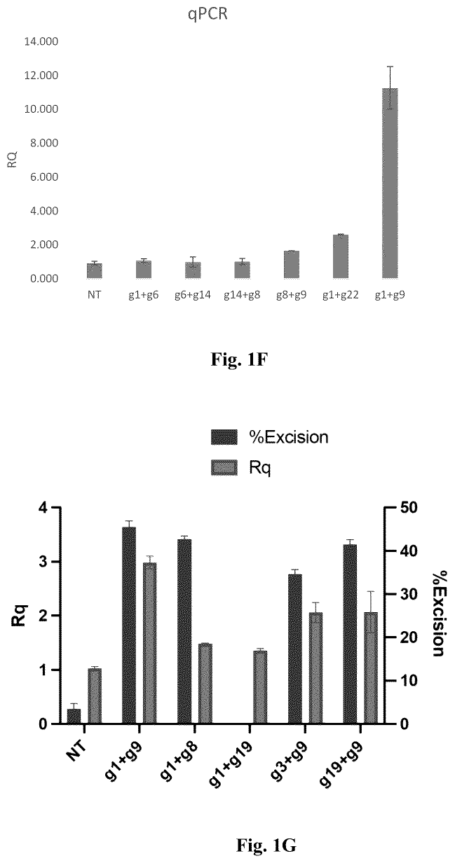

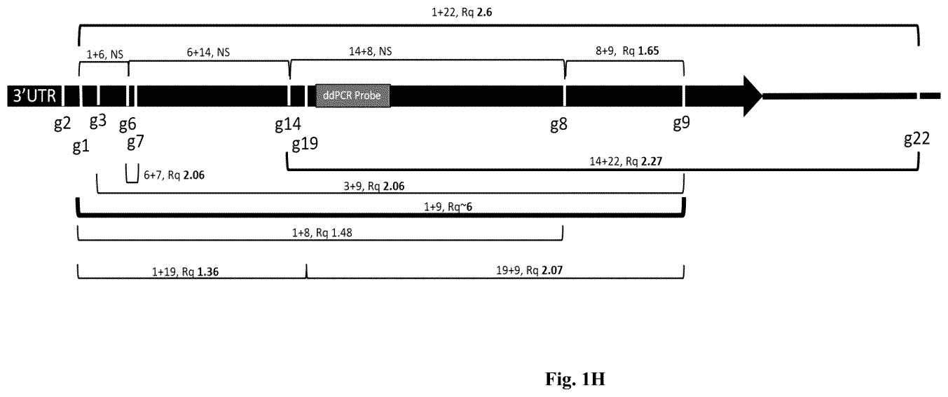

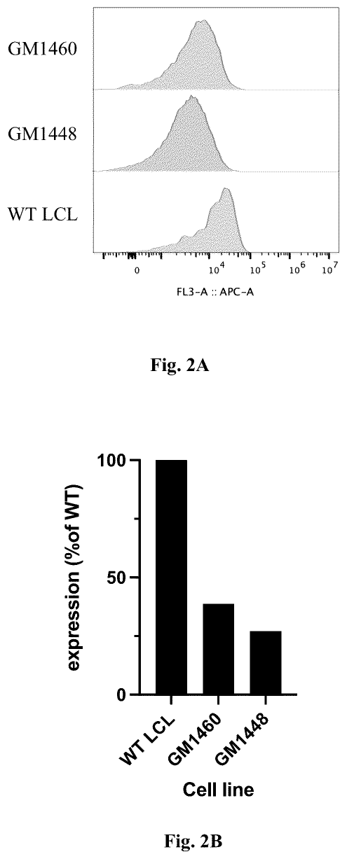

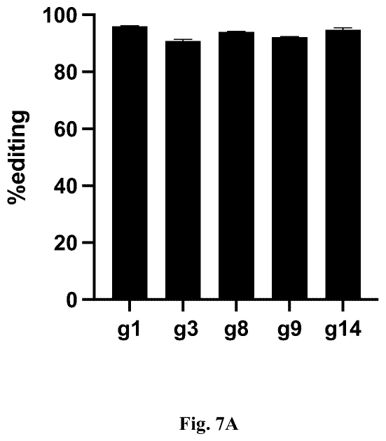

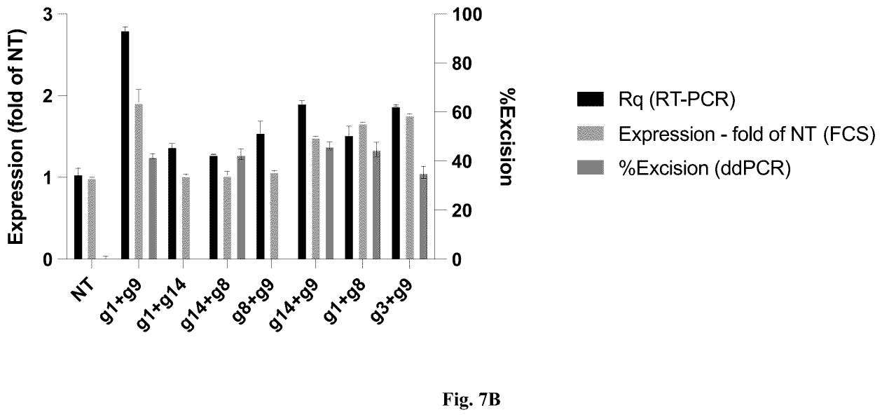

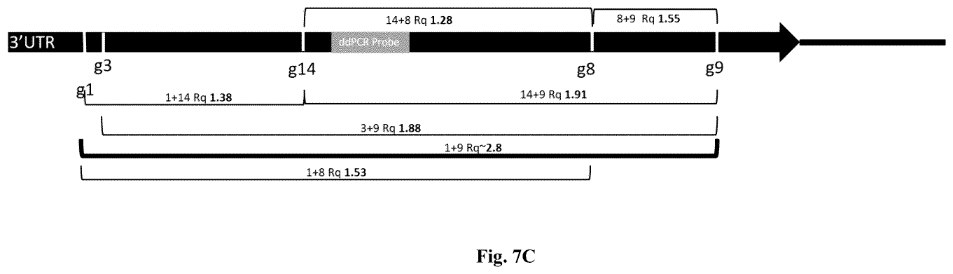

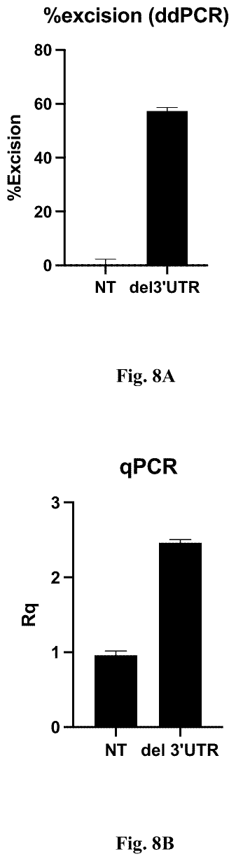

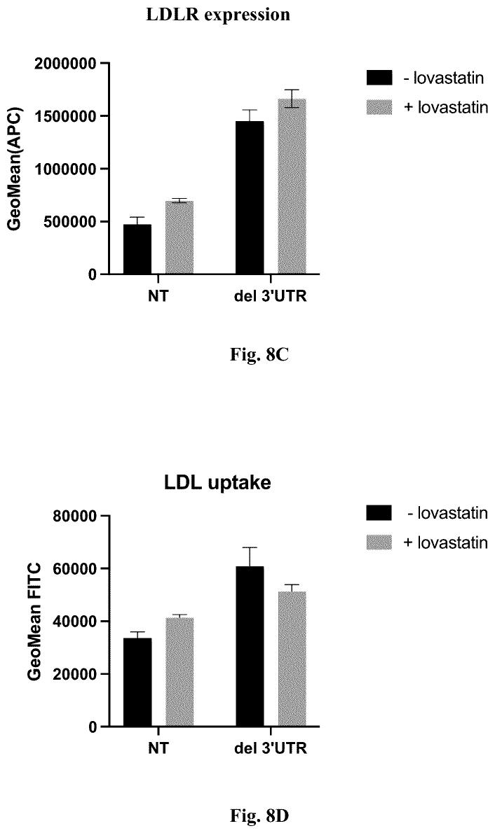

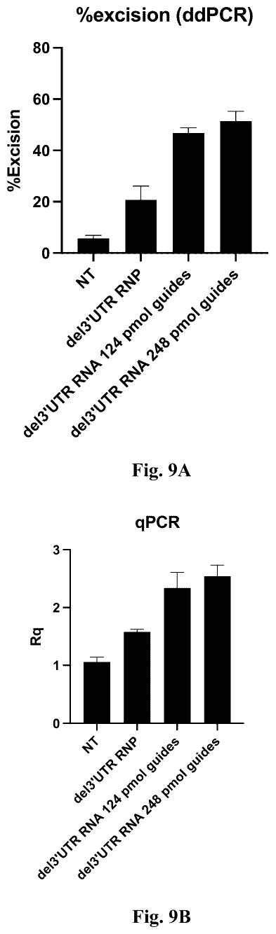

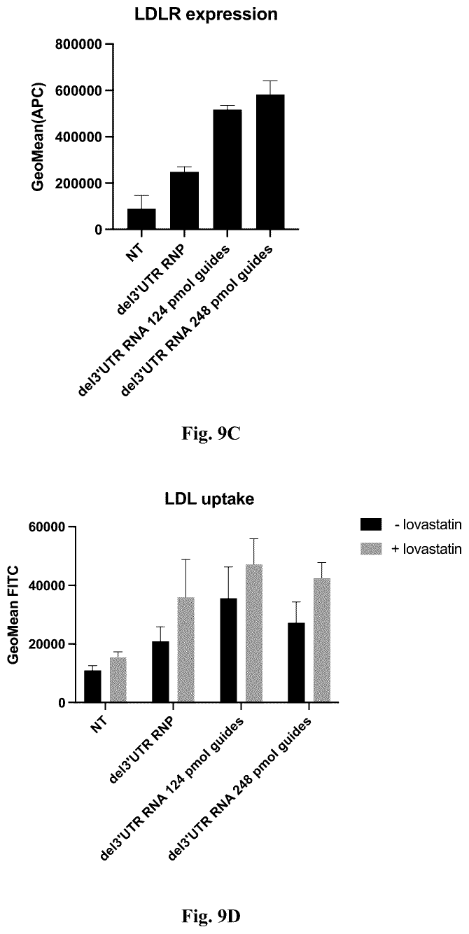

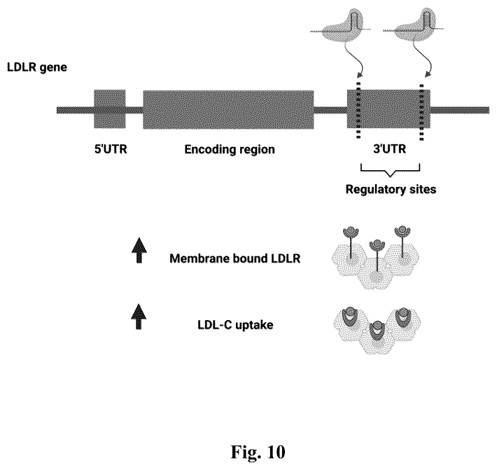

The data presented in this example demonstrates that deletion of the 3′UTR region of the LDLR gene (FIG. 1A) increased the expression of the LDLR gene product both at the RNA and protein levels and increases cholesterol uptake in vitro (FIG. 1B, FIG. 1C, and FIG. 1D). The cellular intake kinetics of fluorescently labeled LDL-cholesterol was measured using flow cytometry in wild-type and 3′UTR-excised HepG2 cells. A two-fold increase in the uptake of cholesterol was observed following 3′ UTR excision at all the time points tested (FIG. 1E). See also FIGS. 1F-1H, FIGS. 7A-7C, FIGS. 8A-8D, and FIGS. 9A-9D.

An ex vivo proof of concept for a LDLR 3′UTR excision strategy was established in FH patient-derived LCL cells, which are EBV-transformed lymphoblastoid B-cell lines. These cells have several advantages in their use as a disease model, such as ease of derivation, facile growth, and genomic stability. The abundance and functionality of LDLR in these cells has also been demonstrated (P. C. Chan et al. 1997).

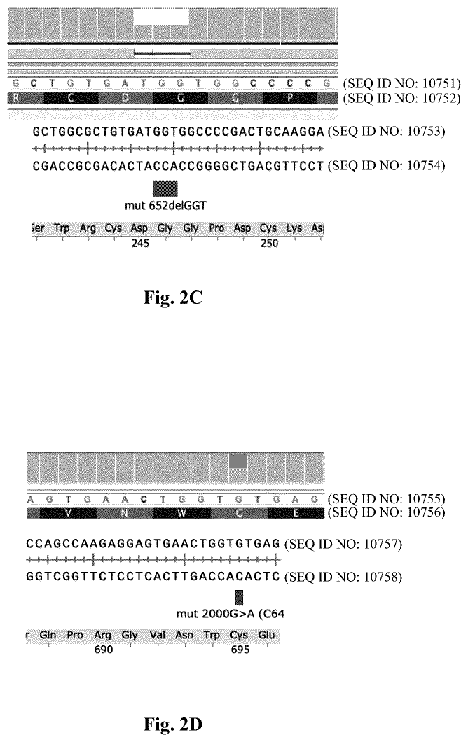

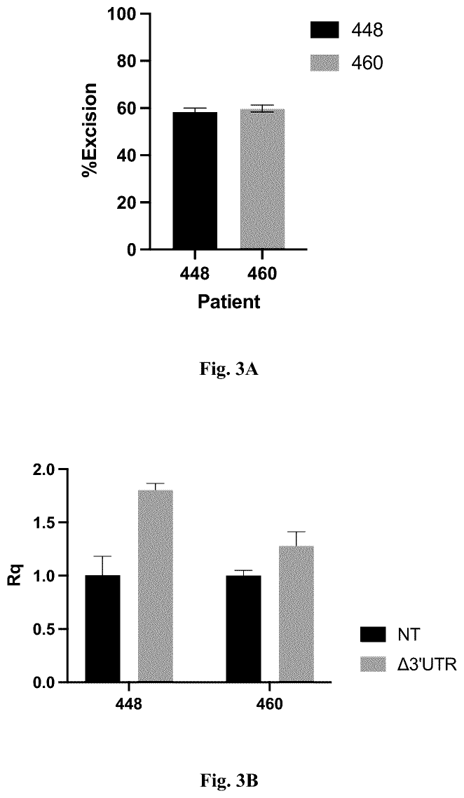

Two cell lines representing FH heterozygous individuals were characterized. The first cell line, GM1448, harbors a ΔG197 mutation, which according to ClinVarMiner is known to be a founder mutation in the Lithuanian Ashkenazi Jewish population and has been observed in 35% of 71 Ashkenazi Jewish families affected by FH. Functional studies have shown that this variant causes defective protein transport to the Golgi complex and the mutant protein shows <2% LDLR activity in cells from a homozygous individual. The second cell line, GM1460, has a missense mutation at C646Y resulting from a 2000G>A substitution. According to ClinVarMiner, this mutation causes reduced processing of LDLR leading to approximately 20% of the receptors being processed to a mature form in homozygous individuals (FIGS. 2A-2D).

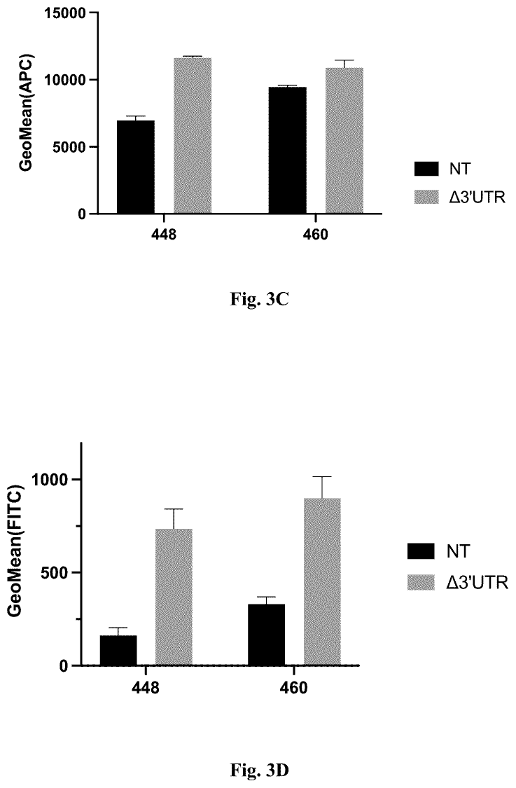

Excision of the LDLR 3′UTR by SpCas9 and specific guide RNA molecules in FH patient LCL cells caused an upregulation of LDLR mRNA, LDLR surface protein, and cholesterol uptake compared to non-treated cells (FIGS. 3A-3D).

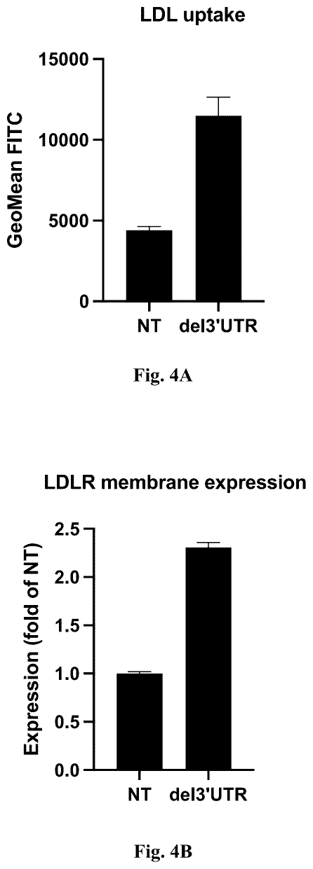

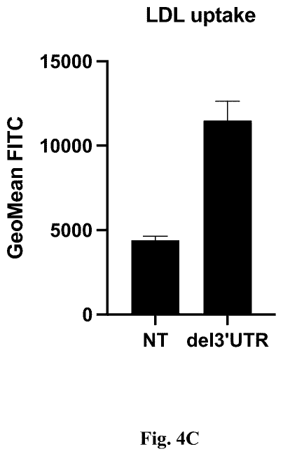

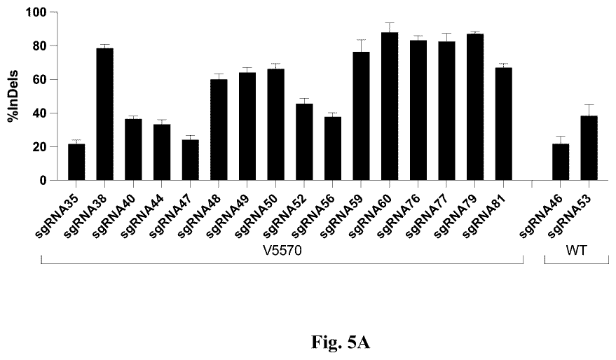

For an animal model selection as an in vivo proof of concept, a 3′UTR excision strategy was validated in mice Hepal-6 hepatocyte cells. Excision of the 3′UTR of the mouse LDLR gene using a mouse editing composition resulted in upregulation of surface LDLR and increases in cholesterol uptake (FIGS. 4A-4C).

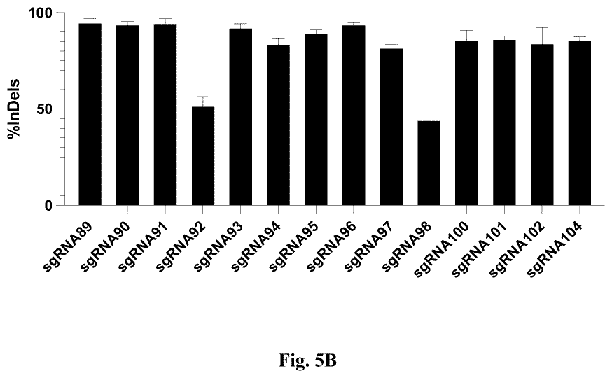

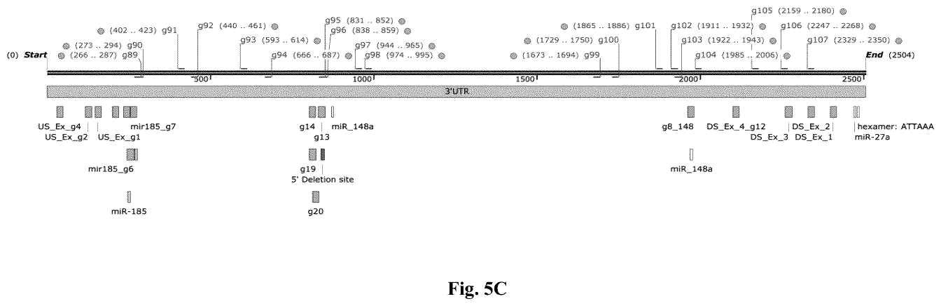

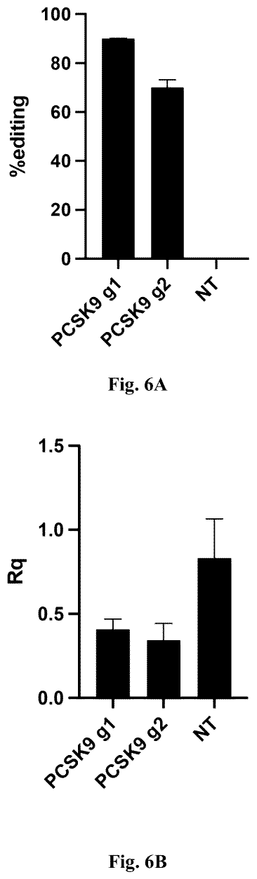

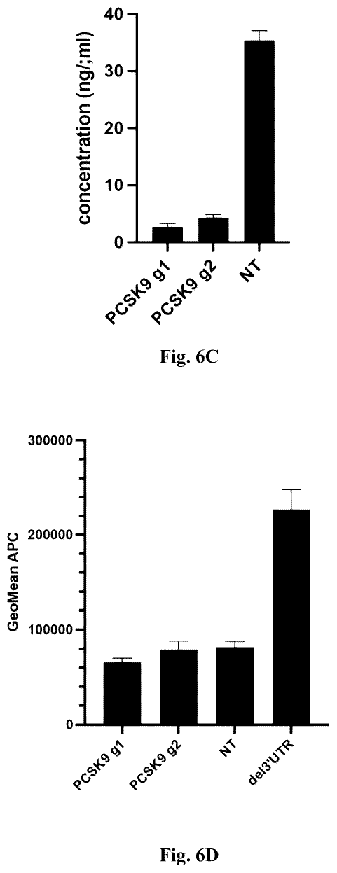

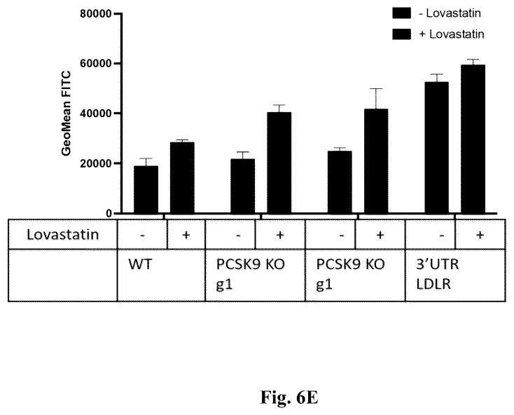

Editing compositions using alternative CRISPR nucleases were also validated for use in LDLR editing. Guide RNA molecules that alter the 3′UTR of LDLR were screened with (1) an OMNI-50 CRISPR nuclease, which is described in PCT International Application Publication No. WO/2020/223513, the content of which is hereby incorporated by reference, (2) an OMNI-79 CRISPR nuclease, which is described in PCT International Application Publication No. WO/2021/248016, the content of which is hereby incorporated by reference, (3) an enhanced activity variant of the OMNI-79 CRISPR nuclease referred to as OMNI-79 V5570, and (4) an OMNI-103 CRISPR nuclease using a DNA transfection method in HeLa cells (FIGS. 5A-5C). It was found that OMNI-79 V5570 improves the editing levels for majority of the sgRNAs tested. Several highly active guides were identified for all tested nucleases tested.

TABLE 2
Alternative OMNI CRISPR nucleases, PAM requirements, and sgRNA
scaffold sequences

| OMNI CRISPR | PAM | |
|---|---|---|
| Nuclease and gRNA | Sequence | sgRNA Scaffold Sequence |
| OMNI-50 | NGG | GUUUGAGAGUUAUGUAAGAAAUUACAUGACGAGUUC |
| (SEQ ID NO: 10749) | | AAAUAAAAAUUUAUUCAAACCGCCUAUUUAUAGGCCG |
| | | CAGAUGUUCUGCAUUAUGCUUGCUAUUGCAAGCUUUU |
| | | UU (SEQ ID NO: 10750) |
| OMNI-79 | NGR | GUUGCCGCUGGAGAAAUCCAGUUGUUAACAAGC |
| (SEQ ID NO: 10744) | | AGCUUGACUGCACCAAAUAAGGCGGGGGCUGCG |
| | | GCCCUCGCUUUUUU (SEQ ID NO: 10747) |
| OMNI-79 V5570 | NGR | GUUGCCGCUGGAGAAAUCCAGUUGUUAACAAGC |
| (SEQ ID NO: 10745) | | AGCUUGACUGCACCAAAUAAGGCGGGGGCUGCG |
| | | GCCCUCGCUUUUUU (SEQ ID NO: 10747) |
| OMNI-103 | NNRACT | GUUUGAGAGUAGUGUAAGAAAUUACACUACAAG |
| (SEQ ID NO: 10746) | | UUCAAAUAAAAAUUUAUUCAAAUCCAUUUGCUA |
| | | CAUUGUGUAGAAUUUAAAGAUCUGGCAACAGAU |
| | | CUUUUUUU (SEQ ID NO: 10748) |

TABLE 3
LDLR-targeting guide sequence portions and CRISPR nucleases utilized

| | | | | | Distance from |
|---|---|---|---|---|---|
| | | | | Human | stop codon/ |
| Nuclease | Guide | | | Genomic | or polyA |
| Utilized | Name | Guide Sequence Portion | PAM | Location | signal (bp) |
| OMNI-79 | sgRNA35 | GGACAGUGCCCAUGCAAU | TGGGTT | 19:11131462 | 170 from stop |
| V5570 or | (g35) | GGCU (SEQ ID NO: 9375) | | | codon |
| OMNI-50 | | | | | |
| OMNI-79 | sgRNA38 | AUCCCAACCCAAGCCAUU | GGGCAC | 19:11131467 | 153 from stop |
| V5570 or | (g38) | GCAU (SEQ ID NO: 7871) | | | codon |
| OMNI-50 | | | | | |
| OMNI-79 | sgRNA40 | AAUCCCAACCCAAGCCAU | TGGGCA | 19:11131468 | 154 from stop |
| V5570 or | (g40) | UGCA (SEQ ID NO: 7340) | | | codon |
| OMNI-50 | | | | | |
| OMNI-79 | sgRNA44 | CCUGUUUCUCUUAUCCUU | AGGAAA | 19:11131508 | 194 from stop |
| V5570 or | (g44) | CACG (SEQ ID NO: 8585) | | | codon |
| OMNI-50 | | | | | |
| OMNI-79 or | sgRNA46 | AGGAUAAGAGAAACAGG | GGGGAC | 19:11131519 | 227 from stop |
| OMNI-50 | (g46) | CCCGG (SEQ ID NO: 7707) | | | codon |
| OMNI-79 | sgRNA47 | GGAUAAGAGAAACAGGC | GGGACC | 19:11131520 | 228 from stop |
| V5570 or | (g47) | CCGGG (SEQ ID NO: 9409) | | | codon |
| OMNI-50 | | | | | |
| OMNI-79 | sgRNA48 | GAUGACACCUCCAUUUCU | AGGAAG | 19:11131550 | 261 from stop |
| V5570 or | (g48) | CUCC (SEQ ID NO: 9133) | | | codon |
| OMNI-50 | | | | | |
| OMNI-79 | sgRNA49 | AAAACUUCCUGGAGAGA | AGGTGT | 19:11131554 | 243 from stop |
| V5570 or | (g49) | AAUGG (SEQ ID NO: 7134) | | | codon |
| OMNI-50 | | | | | |
| OMNI-79 | sgRNA50 | CUCAAAACUUCCUGGAGA | TGGAGG | 19:11131557 | 246 from stop |
| V5570 or | (g50) | GAAA (SEQ ID NO: 8725) | | | codon |
| OMNI-50 | | | | | |
| OMNI-79 | sgRNA52 | AUGUUUGAGGAUUGUGU | TGGAGA | 19:11131589 | 278 from stop |
| V5570 or | (g52) | CACGG (SEQ ID NO: 7963) | | | codon |
| OMNI-50 | | | | | |
| OMNI-79 or | sgRNA53 | UCCAUGUUUGAGGAUUG | CGGTGG | 19:11131592 | 281 from stop |
| OMNI-50 | (g53) | UGUCA (SEQ ID NO: 10059) | | | codon |
| OMNI-79 | sgRNA56 | UUUUCUGUCGUGUGUGU | TGGGAT | 19:11132195 | N/A |
| V5570 or | (g56) | UGGGA (SEQ ID NO: 10723) | | | |
| OMNI-50 | | | | | |
| OMNI-79 | sgRNA59 | UGUUGGGAUGGGAUCCC | AGGGAA | 19:11132209 | N/A |
| V5570 or | (g59) | AGGCC (SEQ ID NO: 10486) | | | |
| OMNI-50 | | | | | |
| OMNI-79 | sgRNA60 | GUUGGGAUGGGAUCCCA | GGGAAA | 19:11132210 | N/A |
| V5570 or | (g60) | GGCCA (SEQ ID NO: 9839) | | | |
| OMNI-50 | | | | | |
| OMNI-79 | sgRNA76 | GAAGCCACUCAUACAUAC | GGGACA | 19:11133699 | 96 from polyA |
| V5570 or | (g76) | AACG (SEQ ID NO: 8967) | | | signal |
| OMNI-50 | | | | | |
| OMNI-79 | sgRNA77 | AGAAGCCACUCAUACAUA | GGGGAC | 19:11133700 | 95 from polyA |
| V5570 or | (g77) | CAAC (SEQ ID NO: 7603) | | | signal |
| OMNI-50 | | | | | |
| OMNI-79 | sgRNA79 | GUUGUAUGUAUGAGUGG | GGGAGA | 19:11133713 | 67 from polyA |
| V5570 or | (g79) | CUUCU (SEQ ID NO: 9843) | | | signal |
| OMNI-50 | | | | | |
| OMNI-79 | sgRNA81 | GUAUGAGUGGCUUCUGG | GGGTGT | 19:11133706 | 60 from polyA |
| V5570 or | (g81) | GAGAU (SEQ ID NO: 9673) | | | signal |
| OMNI-50 | | | | | |
| OMNI-103 | sgRNA89 | GUGUCACGGUGGAGAGA | AAAACT | 19:11131576 | 259 from stop |
| | (g89) | AACUC (SEQ ID NO: 9794) | | | codon |
| OMNI-103 | sgRNA90 | GAGGAUUGUGUCACGGU | GAAACT | 19:11131583 | 265 from stop |
| | (g90) | GGAGA (SEQ ID NO: 9085) | | | codon |
| OMNI-103 | sgRNA91 | ACCAAUCUCUAAGCCAAA | TAAACT | 19:11131718 | 429 from stop |
| | (g91) | CCCC (SEQ ID NO: 7460) | | | codon |
| OMNI-103 | sgRNA92 | UUGCAUAGAAGAGGUAA | TTGACT | 19:11131750 | 433 from stop |
| | (g92) | ACACG (SEQ ID NO: 10613) | | | codon |
| OMNI-103 | sgRNA93 | GAAUCCGUGGUGGCACCG | CAAACT | 19:11131909 | 624 from stop |
| | (g93) | AGAC (SEQ ID NO: 8985) | | | codon |
| OMNI-103 | sgRNA94 | UAAUAAAUAUUAAGGGU | GTGACT | 19:11131976 | 663 from stop |
| | (g94) | GACCA (SEQ ID NO: 9903) | | | codon |
| OMNI-103 | sgRNA95 | AAAUGUACACAGUGUAC | TGAACT | 19:11132141 | N/A |
| | (g95) | AACUC (SEQ ID NO: 7206) | | | |
| OMNI-103 | sgRNA96 | AAAUGCCAAAUGUACAC | ACAACT | 19:11132148 | N/A |
| | (g96) | AGUGU (SEQ ID NO: 7201) | | | |
| OMNI-103 | sgRNA97 | GACAGAGAGGGGCAGGU | GGGACT | 19:11132260 | N/A |
| | (g97) | UGACC (SEQ ID NO: 9007) | | | |
| OMNI-103 | sgRNA98 | AAAGCCGUGAUCGUGAA | AGAACT | 19:11132290 | N/A |
| | (g98) | UAUCG (SEQ ID NO: 7175) | | | |
| OMNI-103 | sgRNA100 | UGCCACCGUACCCAGCUG | TTAACT | 19:11133039 | N/A |
| | (g100) | AUUU (SEQ ID NO: 10301) | | | |
| OMNI-103 | sgRNA101 | AUGAAGACCCUAUUUCA | ACAACT | 19:11133181 | N/A |
| | (g101) | GAAAU (SEQ ID NO: 7904) | | | |
| OMNI-103 | sgRNA102 | CCUCCAGUCUGGAUCGUU | GGGACT | 19:11133227 | N/A |
| | (g102) | UGAC (SEQ ID NO: 8537) | | | |
| OMNI-103 | sgRNA104 | GAGAGACAGUGACAGCC | CAGACT | 19:11133301 | 469 from |
| | (g103) | UCCGU (SEQ ID NO: 9067) | | | poly A signal |
| SpCas9 | sgRNA1 | CAGUGCCCAUGCAAUGGC | GGGTTG | 19:11131463 | 171 from stop |
| | (g1) | UU (SEQ ID NO: 1120) | | | codon |
| SpCas9 | sgRNA2 | AUUCAUCUGGGAGGCAG | AGGCTT | 19:11131433 | 141 from stop |
| | (g2) | AAC (SEQ ID NO: 860) | | | codon |
| SpCas9 | sgRNA3 | AAGGAUAAGAGAAACAG | CGGGGG | 19:11131516 | 224 from stop |
| | (g3) | GCC (SEQ ID NO: 181) | | | codon |
| SpCas9 | sgRNA4 | AACCCUUCCUGAGACCUC | CGGCCT | 19:11131345 | 53 from stop |
| | (g4) | GC (SEQ ID NO: 119) | | | codon |
| SpCas9 | sgRNA6 | CAAAACUUCCUGGAGAG | TGGAGG | 19:11131557 | 243 from stop |
| | (g6) | AAA (SEQ ID NO: 901) | | | codon |
| SpCas9 | sgRNA7 | GAGAGAAACUCAAAACU | TGGAGA | 19:11131567 | 253 from stop |
| | (g7) | UCC (SEQ ID NO: 1927) | | | codon |
| SpCas9 | sgRNA8 | GUUACUGUUGCACUGAU | CGGAGA | 19:11133279 | 502 from |
| | (g8) | GUC (SEQ ID NO: 2656) | | | poly A signal |
| SpCas9 | sgRNA9 | AUGAGUGGCUUCUGGGA | GGGTGT | 19:11133713 | 60 from polyA |
| | (g9) | GAU (SEQ ID NO: 796) | | | signal |
| SpCas9 | sgRNA14 | AGAAAGUGCAAGGAGAC | GGGAAT | 19:11132114 | 800 from stop |
| | (g14) | CAC (SEQ ID NO: 477) | | | codon |
| SpCas9 | sgRNA19 | GAGAAAGUGCAAGGAGA | CGGGAA | 19:11132115 | 801 from stop |
| | (g19) | CCA (SEQ ID NO: 1913) | | | codon |
| SpCas9 | sgRNA20 | CUCUGAACUGAGAAAGU | AGGAGA | 19:11132124 | 810 from stop |
| | (g20) | GCA (SEQ ID NO: 1640) | | | codon |
| SpCas9 | sgRNA22 | UAGGCUUGUGGGACACU | GGGGAT | out of range | Downstream to |
| | (g22) | ACA (SEQ ID NO: 10736) | | | the 3'UTR |
| SpCas9 | sgRNA82 | GGAAGUGGGUAGGGGUC | AGGATG | 19:11132088 | 774 from stop |
| | (g82) | GGG (SEQ ID NO: 2219) | | | codon |
| SpCas9 | sgRNA83 | AAUGGAAGUGGGUAGGG | GGGAGG | 19:11132091 | 777 from stop |
| | (g83) | GUC (SEQ ID NO: 247) | | | codon |
| SpCas9 | sgRNA84 | GAAUGGAAGUGGGUAGG | CGGGAG | 19:11132092 | 778 from stop |
| | (g84) | GGU (SEQ ID NO: 1853) | | | codon |
| SpCas9 | sgRNA85 | CAUCUCUUAAAAAAUGA | TGGCCA | 19:11132889 | N/A |
| | (g85) | AUU (SEQ ID NO: 1151) | | | |
| SpCas9 | sgRNA86 | AAAUGAAUUUGGCCAGA | AGGTGC | 19:11132901 | N/A |
| | (g86) | CAC (SEQ ID NO: 76) | | | |
| SpCas9 | sgRNA1m | GAGCAACACAGAGCCCAG | AGGTGG | N/A (mouse | N/A |
| | (glm) | GG (SEQ ID NO: 10737) | | specific) | |
| SpCas9 | sgRNA6m | GUGCGUUGCUUUGAGUG | GGGAAC | N/A (mouse | N/A |
| | (g6m) | GGU (SEQ ID NO: 10738) | | specific) | |

### Example 3: Summary of Results—CRISPR-Based Gene Editing Enhances LDLR Expression and Boosts LDL-C uptake in Familial Hypercholesterolemia

Familial hypercholesterolemia (FH) is a prevalent autosomal dominant disorder characterized by a lifelong elevation of low-density lipoprotein cholesterol (LDL-C), which results in early-onset atherosclerosis and coronary events. About 85% to 90% of genetically confirmed FH is caused by pathogenic mutations in the LDLR gene, haploinsufficiency of which leads to reduced LDL-C uptake. Lifelong lipid-lowering medications, such as statins and Ezetimibe, are currently available, however, they are often intolerable by patients and fail to attain desired LDL-C levels. A more recent therapeutic approach is based on subcutaneously injected monoclonal antibodies that transiently inhibit Proprotein Convertase Subtilisin/Kexin type 9 (PCSK9), a protein that promotes LDLR lysosomal degradation. Permanent inhibition of PCSK9 by base editing is currently being developed, but the long-lasting consequences of such irreversible knockdown of a gene that is not even the pathogenic basis of FH remain unknown. Additionally, such an approach is frequently insufficient as a monotherapy, and is thus prescribed in combination with a statin therapy.

This disclosure presents a CRISPR-based gene editing strategy for upregulation of LDLR expression by excising at least one portion of the LDLR 3′ UTR, which contains sites that negatively regulate LDLR expression.

This editing strategy was tested in a HepG2 cell line, FH patient-derived lymphoblastoid cell lines (LCLs), and a mouse hepatoma cell line (Hepal-6). Excision of the LDLR 3′UTR was confirmed by ddPCR and LDLR mRNA levels were quantified by qRT-PCR. Total and membrane-bound LDLR levels were determined by western blot and flow cytometry, respectively, using specific antibodies. Finally, the effect of LDLR 3′UTR excision on cholesterol uptake was assessed by measuring the cellular intake of fluorescently labeled LDL-C using flow cytometry.

Excision efficiency in HepG2 cells was about 60%. Excised cells showed a 3- to 6-fold upregulation of LDLR mRNA levels and a 3-fold increase in membrane-bound LDLR as compared to non-treated cells. LDLR 3′UTR excision resulted in a 2-fold increase in the uptake of cholesterol at all time points tested. Patient-derived LCLs showed similar outcomes, with a 3-4-fold increase in LDL-C uptake. An increase in LDLR mRNA levels and membrane protein expression followed by an enhanced LDL-C uptake was also observed in an excised mouse liver cell line, further supporting the future evaluation of the editing strategy in an in vivo mouse model. A comparative analysis showed that the strategy outperforms PCSK9 knockout and statins in increasing cholesterol uptake.

These findings support the CRISPR-based gene editing strategy of truncating regions responsible for rapid LDLR mRNA turnover to enhance LDLR expression and boost LDL-C uptake. This unique approach could be useful for a variety of hypercholesterolemia-related disorders.

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Anzalone et al. (2019) “Search-and-replace genome editing without double-strand brakes or donor DNA”, Nature 576, 149-157.
4. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
5. Behr (1994) “Gene transfer with synthetic cationic amphiphiles: Prospects for gene therapy”, Bioconjuage Chem 5:382-89.
6. Bjornsson et al. (2021) “Lifelong reduction in LDL (low-density lipoprotein) cholesterol due to a gain-of-function mutation in LDLR”, Circulation: Genomic and Precision Medicine, 14:e003029.
7. Blaese (1995) “Vectors in cancer therapy: how will they deliver”, Cancer Gene Ther. 2:291-97.
8. Blaese et al. (1995) “T lympocyte-directed gene therapy for ADA-SCID: initial trial results after 4 years”, Science 270(5235):475-80.
9. Buchschacher and Panganiban (1992) “Human immunodeficiency virus vectors for inducible expression of foreign genes”, J. Virol. 66:2731-39.
10. Burstein et al. (2017) “New CRISPR-Cas systems from uncultivated microbes”, Nature 542:237-41.
11. Chan et al. (1997) “Surface expression of low density lipoprotein receptor in EBV-transformed lymphocytes: characterization and use for studying familial hypercholesterolemia”, Atherosclerosis 131:149-160.
12. Chang and Wilson (1987) “Modification of DNA ends can decrease end-joining relative to homologous recombination in mammalian cells”, Proc. Natl. Acad. Sci. USA 84:4959-4963.
13. Chung et al. (2006) “Agrobacterium is not alone: gene transfer to plants by viruses and other bacteria”, Trends Plant Sci. 11(1):1-4.
14. Coelho et al. (2013) “Safety and efficacy of RNAi therapy for transthyretin amyloidosis” N. Engl. J. Med. 369, 819-829.
15. Collias and Beisel (2021) “CRISPR technologies and the search for the PAM-free nuclease”, Nature Communications 12, 555.
16. Crystal (1995) “Transfer of genes to humans: early lessons and obstacles to success”, Science 270(5235):404-10.
17. Dillon (1993) “Regulation gene expression in gene therapy” Trends in Biotechnology 11(5):167-173.
18. Dranoff et al. (1997) “A phase I study of vaccination with autologous, irradiated melanoma cells engineered to secrete human granulocyte macrophage colony stimulating factor”, Hum. Gene Ther. 8(1): 111-23.
19. Dunbar et al. (1995) “Retrovirally marked CD34-enriched peripheral blood and bone marrow cells contribute to long-term engraftment after autologous transplantation”, Blood 85:3048-57.
20. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.
21. Gao and Huang (1995) “Cationic liposome-mediated gene transfer” Gene Ther. 2(10):710-22.
22. Haddada et al. (1995) “Gene Therapy Using Adenovirus Vectors”, in: The Molecular Repertoire of Adenoviruses III: Biology and Pathogenesis, ed. Doerfler and Böhm, pp. 297-306.
23. Han et al. (1995) “Ligand-directed retro-viral targeting of human breast cancer cells”, Proc Natl Acad Sci U.S.A. 92(21):9747-51.
24. Hu et al. (2018) “Evolved Cas9 variants with broad PAM compatibility and high DNA specificity”, Nature 556(7699): 57-63.
25. Jiang et al. (2015) “The distribution and characteristics of LDL receptor mutations in China: A systematic review”, Scientific Reports 5:17272.
26. Jinek et al. (2012) “A programmable dual-RNA-guided DNA endonuclease in adaptive bacterial immunity,” Science 337(6096): 816-21.
27. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
28. Judge et al. (2006) “Design of noninflammatory synthetic siRNA mediating potent gene silencing in vivo”, Mol Ther. 13(3):494-505.
29. Knouff et al. (2001) “Doubling expression of the low density lipoprotein receptor by truncation of the 3′-untranslated region sequence ameliorates type III hyperlipoproteinemia in mice expressing the human ApoE2 isoform”, JBC 276(9):3856-3862.
30. Kohn et al. (1995) “Engraftment of gene-modified umbilical cord blood cells in neonates with adnosine deaminase deficiency”, Nature Medicine 1:1017-23.
31. Kremer and Perricaudet (1995) “Adenovirus and adeno-associated virus mediated gene transfer”, Br. Med. Bull. 51(1): 31-44.
32. Macdiarmid et al. (2009) “Sequential treatment of drug-resistant tumors with targeted minicells containing siRNA or a cytotoxic drug”, Nat Biotehcnol. 27(7):643-51.
33. Malech et al. (1997) “Prolonged production of NADPH oxidase-corrected granulocyes after gene therapy of chronic granulomatous disease”, PNAS 94(22): 12133-38.
34. Miller et al. (1991) “Construction and properties of retrovirus packaging cells based on gibbon ape leukemia virus”, J Virol. 65(5):2220-24.
35. Miller (1992) “Human gene therapy comes of age”, Nature 357:455-60.
36. Mitani and Caskey (1993) “Delivering therapeutic genes—matching approach and application”, Trends in Biotechnology 11(5): 162-66.
37. Nabel and Felgner (1993) “Direct gene transfer for immunotherapy and immunization”, Trends in Biotechnology 11(5):211-15.
38. Nishimasu et al. (2018) “Engineered CRISPR-Cas9 nuclease with expanded targeting space”, Science 361(6408): 1259-1262.
39. Remy et al. (1994) “Gene Transfer with a Series of Lipphilic DNA-Binding Molecules”, Bioconjugate Chem. 5(6):647-54.
40. Soutar and Naoumova (2007) “Mechanisms of disease: genetic causes of familial hypercholeserolemia”, Nat Clin Pract Cardiovasc Med. 4(4): 214-25.
41. Sentmanat et al. (2018) “A Survey of Validation Strategies for CRISPR-Cas9 Editing”, Scientific Reports 8:888, doi: 10.1038/s41598-018-19441-8.
42. Sommerfelt et al. (1990) “Localization of the receptor gene for type D simian retroviruses on human chromosome 19”, J. Virol. 64(12):6214-20.
43. Van Brunt (1988) “Molecular framing: transgenic animals as bioactors” Biotechnology 6:1149-54.
44. Vigne et al. (1995) “Third-generation adenovectors for gene therapy”, Restorative Neurology and Neuroscience 8(1,2): 35-36.
45. Walton et al. (2020) “Unconstrained genome targeting with near-PAMless engineered CRISPR-Cas9 variants”, Science 368(6488):290-296.
46. Weissman and Kariko (2015) “mRNA:Fulfilling the promise of gene therapy”, Molecular Therapy (9): 1416-7.
47. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
48. Yu et al. (1994) “Progress towards gene therapy for HIV infection”. Gene Ther. 1(1):13-26.
49. Zetsche et al. (2015) “Cpf1 is a single RNA-guided endonuclease of a class 2 CRIPSR-Cas system” Cell 163(3): 759-71.
50. Zuris et al. (2015) “Cationic lipid-mediated delivery of proteins enables efficient protein based genome editing in vitro and in vivo” Nat Biotechnol. 33(1): 73-80.

1. A method for increasing the endogenous expression of a Low-Density Lipoprotein Receptor (LDLR) gene in a cell, the method comprising modifying the LDLR gene or a transcript encoded by the LDLR gene.

2. The method of claim 1, wherein the LDLR gene is modified by a CRISPR nuclease, a meganuclease, a transcription activator-like effector nucleases (TALEN), or a zinc finger nuclease (ZFN), or wherein a transcript encoded by the LDLR gene is modified by a small interfering RNA (siRNA) , short hairpin RNA (shRNA) , or microRNA (miRNA) molecule; and/or
wherein the 3′ untranslated region (UTR) of the LDLR gene or the transcript encoded by the LDLR gene is modified; and/or
wherein a miRNA binding site or AU Rich region (AUR) of the LDLR gene in the cell is subjected to an insertion or deletion mutation; and/or
wherein the increase of the expression of an endogenous LDLR gene in a cell is measured by an at least 108, at least 25%, at least 508, at least 758, at least 100%, at least 1508, at least 2008, at least 2508, at least 3008, at least 3508, at least 400%, at least 4508, at least 500% increase, at least 550% increase, or at least 600% increase in LDLR transcript levels or LDLR protein levels relative to LDLR transcript levels or LDLR protein levels in the cell prior to modification of the LDLR gene; and/or wherein the increase of the expression of an endogenous LDLR gene results in an at least 108, at least 25%, at least 50%, at least 75%, at least 100%, at least 150%, at least 200%, at least 250%, at least 300%, at least 350%, at least 400%, at least 450%, or at least 500% increase in LDL-C uptake by the cell relative to the LDL-C uptake by the cell prior to modification of the LDLR gene.

3. (canceled)

4. The method of claim 2, wherein the 3′UTR of the LDLR gene is modified by excision of the 3′ UTR or a portion thereof.

5. The method of claim 1, the method comprises
introducing to the cell a composition comprising:
at least one CRISPR nuclease, or a nucleotide molecule encoding a CRISPR nuclease; and
an RNA molecule, or a DNA molecule encoding the RNA molecule, comprising a guide sequence portion,
wherein a complex of the CRISPR nuclease and the RNA molecule affects a double strand break in an allele of the LDLR gene, thereby increasing the expression of the LDLR gene.

6. The method of claim 5,
wherein the composition is introduced to a cell in a subject or to a cell in culture; and/or
wherein the cell is a liver cell, hepatocyte, or stem cell.

7. (canceled)

8. The method of claim 5,
wherein the CRISPR nuclease, or nucleotide molecule encoding the CRISPR nuclease, and the RNA molecule, or DNA molecule encoding the RNA molecule, are introduced to the cell at substantially the same time or at different times; and/or
wherein the guide sequence portion of the RNA molecule targets a site in the LDLR 3′UTR located at least 30 nucleotides downstream to the stop codon or at least 30 nucleotides upstream to the polyadenylation signal; and/or
wherein the guide sequence portion of the RNA molecule targets a miRNA binding site in a LDLR allele; and/or
wherein guide sequence portion of the RNA molecule targets a miR-85 or miR-148 binding site; and/or
wherein a complex of the CRISPR nuclease and the RNA molecule affects a double strand break in both alleles of the LDLR gene; and/or
wherein the guide sequence portion of the RNA molecule comprises 17-50 contiguous nucleotides containing nucleotides in the sequence set forth in any one of SEQ ID NOs: 1-10736; and/or
wherein the composition introduced to the cell further comprises a second RNA molecule, or a DNA sequence encoding the second RNA molecule, comprising a guide sequence portion; and/or wherein the guide sequence portion of the first RNA molecule or second RNA molecule comprises 1, 2, 3, 4, or 5 nucleotide mismatches relative to a fully complementary LDLR target sequence.

9-12. (canceled)

13. The method of claim 1,
wherein a miR seed sequence is inactivated as a result of the modifying of the LDLR gene.

14. The method of claim 13, wherein the miR seed sequence is UGGUGCUA or CACUGUG; or wherein the miR seed sequence is a miR-85 or miR-148 seed sequence.

15-18. (canceled)

19. The method of claim 8, wherein the composition introduced to the cell further comprises a second RNA molecule, or a DNA sequence encoding the second RNA molecule, comprising a guide sequence portion, and
wherein the guide sequence portion of the second RNA molecule targets a site in the LDLR 3′UTR located at least 30 nucleotides downstream to the stop codon or at least 30 nucleotides upstream to the polyadenylation signal; and/or
wherein the guide sequence portion of the second RNA molecule comprises 17-50 contiguous nucleotides containing nucleotides in the sequence set forth in any one of SEQ ID NOS: 1-10736 other than the sequence of the first RNA molecule; and/or
wherein a LDLR 3′UTR or a portion thereof is excised by a double strand break formed by a CRISPR nuclease and the first RNA molecule and a double strand break formed by a CRISPR nuclease and the second RNA molecule.

20-21. (canceled)

22. The method of claim 19, wherein a LDLR 3′UTR or a portion thereof is excised by a double strand break formed by a CRISPR nuclease and the first RNA molecule and a double strand break formed by a CRISPR nuclease and the second RNA molecule and wherein the endogenous LDLR polyadenylation signal remains intact.

23. (canceled)

24. The method of claim 23, wherein the guide sequence portion of the first RNA molecule or second RNA molecule comprises 1, 2, 3, 4, or 5 nucleotide mismatches relative to a fully complementary LDLR target sequence, and
wherein the guide sequence portion comprises 17-50 contiguous nucleotides containing nucleotides in the sequence set forth in any one of SEQ ID NOs: 1-10736 modified to contain 1, 2, 3, 4, or 5 nucleotide mismatches relative to a fully complementary LDLR target sequence; and/or
wherein the guide sequence portion provides higher targeting specificity to the complex of the CRISPR nuclease and the first RNA molecule relative to a guide sequence portion that has higher complementarity to the mutant allele of the LDLR gene.

25-27. (canceled)

28. A composition comprising an RNA molecule which comprises 17-50 contiguous nucleotides containing nucleotides in the sequence set forth in any one of SEQ ID NOs: 1-10736, or any one of SEQ ID NOS: 1-10736 modified to contain 1, 2, 3, 4, or 5 nucleotide mismatches relative to a fully complementary LDLR target sequence, or a DNA molecule encoding the RNA molecule.

29. The composition of claim 28, further comprising a CRISPR nuclease, or a nucleotide molecule encoding a CRISPR nuclease; and/or
further comprising a tracrRNA molecule; and/or
further comprising a second RNA molecule, or a DNA molecule encoding the second RNA molecule, comprising a guide sequence portion.

30-31. (canceled)

32. The composition of claim 28, further comprising a second RNA molecule, or a DNA molecule encoding the second RNA molecule, comprising a guide sequence portion, and
wherein a guide sequence portion of the second RNA targets a site in the LDLR 3′UTR located at least 30 nucleotides downstream to the stop codon or at least 30 nucleotides upstream to the polyadenylation signal; and/or
wherein the guide sequence portion of the second RNA molecule comprises 17-50 contiguous nucleotides containing nucleotides in the sequence set forth in any one of SEQ ID NOS: 1-10736 other than the sequence of the first RNA molecule, or any one of SEQ ID NOS: 1-10736 other than the sequence of the first RNA molecule modified to contain 1, 2, 3, 4, or 5 nucleotide mismatches relative to a fully complementary LDLR target sequence.

33. (canceled)

34. A cell modified by the method of claim 1.

35. A medicament comprising the composition of claim 28 for use in inactivating a LDLR allele in a cell, wherein the medicament is administered by delivering to the cell the composition of claim 28.

36. A method of treating, ameliorating, or preventing hypercholesterolemia, comprising delivering the composition of claim 28 to a subject experiencing or at risk of experiencing hypercholesterolemia.

37. A method of treating, ameliorating, or preventing hypercholesterolemia, wherein the modified cell of claim 34 is administered to a subject experiencing or at risk of experiencing hypercholesterolemia.

38. A kit for increasing LDLR expression in a cell, comprising the composition of claim 28 and instructions for delivering the composition to the cell.

39. A kit for treating or preventing hypercholesterolemia in a subject, comprising the composition of claim 28 and instructions for delivering the composition or modified cell to a subject experiencing or at risk of experiencing hypercholesterolemia.
